(19) United States
(12) Patent Application Publication
(10) Pub. No.: US 2019/0216800 A1
(43) Pub. Date: Jul. 18, 2019

(54) DOSAGE FORMS AND METHODS FOR ENANTIOMERICALLY ENRICHED OR PURE BUPROPION

(71) Applicant: AXSOME THERAPEUTICS, INC., New York, NY (US)

(72) Inventor: Herriot Tabuteau, New York, NY (US)

(21) Appl. No.: 16/364,005

(22) Filed: Mar. 25, 2019

Related U.S. Application Data
(63) Continuation-in-part of application No. 16/290,653, filed on Mar. 1, 2019.
Application No. 16/290,653 is a continuation-in-part of application No. 15/933,075, filed on Mar. 22, 2018, now Pat. No. 10,251,879.
Application No. 15/933,075 is a continuation of application No. 15/216,545, filed on Jul. 21, 2016, now abandoned.
Application No. 15/216,545 is a continuation-in-part of application No. PCT/US15/28901, filed on May 1, 2015.
Application No. PCT/US15/28901 is a continuation of application No. 14/628,062, filed on Feb. 20, 2015, now Pat. No. 9,402,844.
Application No. 14/628,062 is a continuation of application No. 14/617,624, filed on Feb. 9, 2015, now Pat. No. 9,486,450.
Application No. 14/617,624 is a continuation of application No. 14/604,397, filed on Jan. 23, 2015, now Pat. No. 9,168,234.
Application No. 14/604,397 is a continuation of application No. 14/602,177, filed on Jan. 21, 2015, now Pat. No. 9,402,843.
Application No. 14/602,177 is a continuation of application No. 14/555,085, filed on Nov. 26, 2014, now Pat. No. 9,238,032.
Application No. 14/555,085 is a continuation of application No. 14/554,947, filed on Nov. 26, 2014, now abandoned.
Application No. 14/554,947 is a continuation of application No. 14/554,988, filed on Nov. 26, 2014, now Pat. No. 9,205,083.
Application No. 14/554,988 is a continuation of application No. 14/550,618, filed on Nov. 21, 2014, now Pat. No. 9,198,905.
Application No. 15/216,545 is a continuation-in-part of application No. 15/057,983, filed on Mar. 1, 2016, now Pat. No. 9,408,815.
Application No. 15/057,983 is a continuation-in-part of application No. 14/863,284, filed on Sep. 23, 2015, now Pat. No. 9,278,095.
Application No. 15/216,545 is a continuation-in-part of application No. 14/997,316, filed on Jan. 15, 2016, now Pat. No. 9,457,025.
Application No. 14/997,316 is a continuation-in-part of application No. 14/555,085, filed on Nov. 26, 2014, now Pat. No. 9,238,032.
Application No. 14/555,085 is a continuation of application No. 14/550,618, filed on Nov. 21, 2014, now Pat. No. 9,198,905.
Application No. 14/550,618 is a continuation-in-part of application No. PCT/US2014/064184, filed on Nov. 5, 2014.
Application No. 16/290,653 is a continuation-in-part of application No. 16/116,393, filed on Aug. 29, 2018.
Application No. 16/116,393 is a continuation of application No. 15/621,882, filed on Jun. 13, 2017, now Pat. No. 10,105,361.
Application No. 15/621,882 is a continuation-in-part of application No. 15/164,767, filed on May 25, 2016, now Pat. No. 9,700,528.
Application No. 15/164,767 is a continuation-in-part of application No. 14/628,062, filed on Feb. 20, 2015, now Pat. No. 9,402,844.
Application No. 14/628,062 is a continuation of application No. 14/604,397, filed on Jan. 23, 2015, now Pat. No. 9,168,234.
Application No. 14/604,397 is a continuation of application No. 14/602,177, filed on Jan. 21, 2015, now Pat. No. 9,402,843.
Application No. 14/602,177 is a continuation-in-part of application No. 14/550,618, filed on Nov. 21, 2014, now Pat. No. 9,198,905.
Application No. 15/621,882 is a continuation of application No. 15/280,938, filed on Sep. 29, 2016, now Pat. No. 9,707,191.
Application No. 15/280,938 is a continuation of application No. PCT/US19/19445, filed on Feb. 25, 2019.
(60) Provisional application No. 61/900,354, filed on Nov. 5, 2013.
(60) Provisional application No. 62/336,533, filed on May 13, 2016.
(60) Provisional application No. 62/313,620, filed on Mar. 25, 2016.
(60) Provisional application No. 62/323,438, filed on Apr. 15, 2016.
(60) Provisional application No. 62/313,067, filed on Mar. 24, 2016.
(60) Provisional application No. 62/634,718, filed on Feb. 23, 2018.
(60) Provisional application No. 62/794,469, filed on Jan. 18, 2019.
(60) Provisional application No. 62/809,480, filed on Feb. 22, 2019.

(51) Int. Cl. A61K 31/485; A61K 9/00; A61K 31/137; A61K 31/138; A61K 31/4748

(52) U.S. Cl. CPC A61K 31/485; A61K 9/0053; A61K 31/4748; A61K 31/138; A61K 31/137

(57) ABSTRACT

Described herein are dosage forms of enantiomerically enriched (S)-bupropion or enantiomerically enriched (R)-bupropion. The (S)-bupropion or the (R)-bupropion may be deuterium enriched, or may have natural isotopic abundance. These dosage forms may be administered, either fed or fasted, to treat a condition recited herein, to achieve a certain pharmacokinetic parameter of a bupropion or a metabolite of a bupropion, and/or to enhance dextromethorphan plasma levels.

### CROSS-REFERENCE TO RELATED APPLICATIONS

This application is a continuation-in-part of U.S. patent application Ser. No. 16/290,653, filed Mar. 1, 2019; which is a continuation-in-part of U.S. patent application Ser. No. 15/933,075, filed Mar. 22, 2018; which is a continuation of U.S. patent application Ser. No. 15/216,545, filed Jul. 21, 2016, now abandoned; which is continuation-in-part of International Pat. App. No. PCT/US2015/028901, filed May 1, 2015; which is a continuation of U.S. patent application Ser. No. 14/628,062, filed Feb. 20, 2015, now U.S. Pat. No. 9,402,844; U.S. patent application Ser. No. 14/617,624, filed Feb. 9, 2015, now U.S. Pat. No. 9,486,450; U.S. patent application Ser. No. 14/604,397, filed Jan. 23, 2015, now U.S. Pat. No. 9,168,234; U.S. patent application Ser. No. 14/602,177, filed Jan. 21, 2015, now U.S. Pat. No. 9,402,843; U.S. patent application Ser. No. 14/555,085, filed Nov. 26, 2014, now U.S. Pat. No. 9,238,032; U.S. patent application Ser. No. 14/554,947, filed Nov. 26, 2014, now abandoned; U.S. patent application Ser. No. 14/554,988, filed Nov. 26, 2014, now U.S. Pat. No. 9,205,083; and U.S. patent application Ser. No. 14/550,618, filed Nov. 21, 2014, now U.S. Pat. No. 9,198,905; U.S. patent application Ser. No. 15/216,545 is also a continuation-in-part of U.S. patent application Ser. No. 15/057,983, filed Mar. 1, 2016, now U.S. Pat. No. 9,408,815; which is a continuation-in-part of U.S. patent application Ser. No. 14/863,284, filed Sep. 23, 2015, now U.S. Pat. No. 9,278,095; U.S. patent application Ser. No. 15/216,545 is also a continuation-in-part of U.S. patent application Ser. No. 14/997,316, filed Jan. 15, 2016, now U.S. Pat. No. 9,457,025; which is a continuation-in-part of U.S. patent application Ser. No. 14/555,085, filed Nov. 26, 2014, now U.S. Pat. No. 9,238,032; which is a continuation of U.S. patent application Ser. No. 14/550,618, filed Nov. 21, 2014, now U.S. Pat. No. 9,198,905; which is a continuation-in-part of International Pat. App. No. PCT/US2014/064184, filed Nov. 5, 2014; which claims the benefit of U.S. Prov. Pat. App. No. 61/900,354, filed Nov. 5, 2013; U.S. patent application Ser. No. 15/216,545 also claims the benefit of U.S. Prov. Pat. App. Nos. 62/336,533, filed May 13, 2016; 62/313,620, filed Mar. 25, 2016; 62/323,438, filed Apr. 15, 2016; and 62/313,067, filed Mar. 24, 2016; U.S. patent application Ser. No. 16/290,653 is also a continuation-in-part of U.S. patent application Ser. No. 16/116,393, filed Aug. 29, 2018; which is a continuation of U.S. patent application Ser. No. 15/621,882, filed Jun. 13, 2017, now U.S Pat. No. Ser. No. 10,105,361; which is a continuation-in-part of U.S. patent application Ser. No. 15/164,767, filed May 25, 2016, now U.S. Pat. No. 9,700,528; which is a continuation-in-part of U.S. patent application Ser. No. 14/628,062, filed Feb. 20, 2015, now U.S. Pat. No. 9,402,844; which is a continuation of U.S. patent application Ser. No. 14/604,397, filed Jan. 23, 2015, now U.S. Pat. No. 9,168,234; which is a continuation of U.S. patent application Ser. No. 14/602,177, filed Jan. 21, 2015, now U.S. Pat. No. 9,402,843; which is a continuation-in-part of the above U.S. patent application Ser. No. 14/550,618; the above U.S. patent application Ser. No. 15/621,882 is also a continuation of U.S. patent application Ser. No. 15/280,938, filed Sep. 29, 2016, now U.S. Pat. No. 9,707,191; this application is also a continuation of PCT/US2019/019445, filed Feb. 25, 2019, which claims the benefit of U.S. Prov. Pat. App. Nos. 62/634,718, filed Feb. 23, 2018; 62/794,469, filed Jan. 18, 2019; and 62/809,480, filed Feb. 22, 2019, the entire contents of each of these U.S. patent applications, and each of the U.S. patents, and each of the U.S. publications issued therefrom, are herein incorporated by reference.

### SUMMARY

Described herein are dosage forms of enantiomerically enriched (S)-bupropion or enantiomerically enriched (R)-bupropion. The (S)-bupropion or the (R)-bupropion may be deuterium enriched, or may have natural isotopic abundance. These dosage forms may be administered, either fed or fasted, to treat a condition recited herein, to achieve a certain pharmacokinetic parameter of a bupropion or a metabolite of a bupropion, and/or to enhance dextromethorphan plasma levels.

Some embodiments include a method of delivering a bupropion or a metabolite thereof to plasma comprising orally administering a dosage form containing about 50 mg to about 100 mg of (S)-bupropion that is at least 95% enantiomerically pure, at least once per day, to a human being.

Some embodiments include a method of providing a bupropion to the plasma of a human being, comprising: selecting a human patient in need of the bupropion with a pharmacokinetic profile provided by orally administering a reference dosage form containing a first amount of racemic bupropion at a first dosing frequency; and orally administering a dosage form containing a second amount of (S)-bupropion that is at least 95% enantiomerically pure at the first dosing frequency to achieve the same pharmacokinetic profile that would be achieved by administering the reference dosage form at the first dosing frequency; wherein the first dosing frequency is once daily or twice daily; and wherein the second amount is about 40% to about 60% of the first amount.

Some embodiments include a method of treating a condition that is treatable with racemic bupropion, comprising: selecting a human patient having the condition that is treatable by orally administering a reference dosage form containing a first amount of racemic bupropion at a first dosing frequency; and orally administering a dosage form containing a second amount of (S)-bupropion that is at least 95% enantiomerically pure at the first dosing frequency to achieve the same therapeutic effect that would be achieved by administering the reference dosage form at the first dosing frequency; wherein the first dosing frequency is once daily or twice daily; and wherein the second amount is about 40% to about 60% of the first amount.

Some embodiments include a method of enhancing the plasma levels of (S)-bupropion and dextromethorphan, comprising orally co-administering, at least once per day, dextromethorphan and at least about 90 mg of (S)-bupropion that is at least 95% enantiomerically pure, to a human being in need of treatment with both (S)-bupropion and dextromethorphan, wherein the method achieves a Cₘₐₓ of (S)-bupropion that is at least about 90 ng/mL in the human being, wherein the method is effective in increasing the Cₘₐₓ of (S)-bupropion at least 3-fold as compared to the Cₘₐₓ of (R)-bupropion that results from administering the same amount of (R)-bupropion to the human being.

Some embodiments include a method of enhancing the plasma levels of (R,R)-hydroxybupropion and dextromethorphan, comprising orally co-administering, at least once per day, dextromethorphan and at least about 90 mg of (S)-bupropion that is at least 95% enantiomerically pure, to a human being in need of treatment with both (R,R)-hydroxybupropion and dextromethorphan, wherein the method achieves a Cₘₐₓ of (R,R)-hydroxybupropion that is at least about 700 ng/mL in the human being, wherein the method is effective in increasing the Cₘₐₓ of (R,R)-hydroxybupropion at least 3-fold as compared the Cₘₐₓ of (R,R)-hydroxybupropion that results from administering the same amount of (R)-bupropion to the human being.

Some embodiments include a method of enhancing the plasma levels of (S)-bupropion comprising orally administering, at least once per day, at least about 90 mg of (S)-bupropion that is at least 95% enantiomerically pure, to a human being in need of treatment (S)-bupropion, wherein the method achieves a Cₘᵢₙ of (S)-bupropion that is at least about 20 ng/mL, wherein the method is effective in increasing the Cₘᵢₙ of (S)-bupropion at least 3-fold as compared to the Cₘᵢₙ of (R)-bupropion that results from administering a dosage form containing the same amount of (R)-bupropion to the human being.

Some embodiments include a method of enhancing the plasma levels of (R,R)-hydroxybupropion comprising orally administering, at least once per day, at least about 90 mg of (S)-bupropion that is at least 95% enantiomerically pure, to a human being in need of treatment with (R,R)-hydroxybupropion, wherein the method achieves a Cₘᵢₙ of (R,R)-hydroxybupropion that is at least about 700 ng/mL in the human being, wherein the method is effective in increasing the Cₘᵢₙ of (R,R)-hydroxybupropion at least 3-fold as compared to the Cₘᵢₙ of (R,R)-hydroxybupropion that results from administering the same amount of (R)-bupropion to the human being.

Some embodiments include a method of treating a central nervous system (CNS) disorder in a human being comprising administering: a dosage form comprising a therapeutically effective amount of at least about 95% enantiomerically pure (S)-bupropion, and dextromethorphan, to the human being.

Some embodiments include a method of achieving an increased plasma level of (S)-bupropion while enhancing dextromethorphan plasma levels, comprising administering a dosage form comprising a therapeutically effective amount of at least about 95% enantiomerically pure (S)-bupropion, and dextromethorphan, to a human being in need of treatment with bupropion.

Some embodiments include a dosage form comprising at least about 95% enantiomerically pure (R)-bupropion and dextromethorphan, wherein orally administering the dosage form to a human being provides an increased enhancement to a plasma level of dextromethorphan in the human being as compared to orally administering a reference dosage form containing the same amount of (S)-bupropion and the same amount of dextromethorphan.

Some embodiments include a method of increasing enhancement of dextromethorphan plasma level in a human being, comprising administrating a dosage form comprising at least about 95% enantiomerically pure (R)-bupropion and dextromethorphan to the human being, wherein the dosage form provides an increased enhancement to a plasma level of dextromethorphan in the human being as compared to a reference oral dosage form containing the same amount of (S)-bupropion and the same amount of dextromethorphan.

Some embodiments include a method of treating a central nervous system (CNS) disorder in a human being comprising administering a dosage form comprising a therapeutically effective amount of at least about 95% enantiomerically pure (S)-bupropion to the human being to treat the CNS disorder, wherein the human being does not receive dextromethorphan.

Some embodiments include a method of enhancing the plasma level of (S)-bupropion, comprising administering a dosage form comprising a therapeutically effective amount of at least about 95% enantiomerically pure (S)-bupropion, wherein the dosage form is free of dextromethorphan, to a human being in need of treatment with bupropion.

Some embodiments include a method of enhancing the plasma levels of (S)-bupropion, comprising orally administering an oral dosage form containing at least about 90 mg of (S)-bupropion that is at least 95% enantiomerically pure, to a human being in need of treatment with (S)-bupropion and dextromethorphan, wherein the method achieves a Cₘₐₓ of (S)-bupropion that is at least about 90 ng/mL in the human being, wherein the method is effective in increasing the Cₘₐₓ of (S)-bupropion at least 3-fold as compared to the Cₘₐₓ of (R)-bupropion that results from administering a dosage form containing the same amount of (R)-bupropion to the human being, wherein the human being does not receive dextromethorphan.

Some embodiments include a method of enhancing the plasma levels of (R,R)-hydroxybupropion, comprising orally administering an oral dosage form containing at least about 90 mg of (S)-bupropion that is at least 95% enantiomerically pure, to a human being in need of treatment with (R,R)-hydroxybupropion and dextromethorphan, wherein the method achieves a Cₘₐₓ of (R,R)-hydroxybupropion that is at least about 90 ng/mL, wherein the method is effective in increasing the Cₘₐₓ of (R,R)-hydroxybupropion at least 3-fold as compared to the Cₘₐₓ of (R,R)-hydroxybupropion that results from administering a dosage form containing the same amount of (R)-bupropion to the human being, wherein the human being does not receive dextromethorphan.

Some embodiments include an oral dosage form comprising (R)-bupropion in an enantiomeric excess of at least 95%, and dextromethorphan, wherein the dosage form provides an increased enhancement to a plasma level of dextromethorphan in a human being as compared to a reference oral dosage form containing the same amount of (S)-bupropion and the same amount of dextromethorphan.

Some embodiments include an oral dosage form comprising (R)-bupropion and dextromethorphan, wherein the dosage form can enhance plasma levels of dextromethorphan in a human being on day 1 or day 8 in a much greater extent than that of a reference oral dosage form when orally administered to the human being daily for at least 8 consecutive days, wherein the reference oral dosage form contains the same amount of bupropion as a racemic-bupropion and the same amount of dextromethorphan.

Some embodiments include an oral dosage form comprising (R)-bupropion and dextromethorphan, wherein the dosage form increases a mean Cₘₐₓ of dextromethorphan in a human being on day 1 by at least 150% when orally administered to the human being daily for at least 8 consecutive days as compared to that of a reference oral dosage form on day 1 when orally administered to the human being daily for at least 8 consecutive days, wherein the reference oral dosage form contains the same amount of bupropion as a (S)-bupropion and the same amount of dextromethorphan.

Some embodiments include an oral dosage form comprising (R)-bupropion and dextromethorphan, wherein the dosage form increases a mean Cₘₐₓ of dextromethorphan in a human being on day 8 by at least 25% when orally administered to the human being daily for at least 8 consecutive days as compared to that of a reference oral dosage form on day 8 when orally administered to the human being daily for at least 8 consecutive days, wherein the reference oral dosage form contains the same amount of bupropion as a (S)-bupropion and the same amount of dextromethorphan.

Some embodiments include an oral dosage form comprising (R)-bupropion and dextromethorphan, wherein the dosage form increases a mean Cₘₐₓ of dextromethorphan in a human being on day 8 by at least 20-fold as compared to that of a reference dosage form on day 1, when orally administered to the human being daily for at least 8 consecutive days, wherein the reference oral dosage form contains the same amount of bupropion as a racemic-bupropion and the same amount of dextromethorphan.

Some embodiments include an oral dosage form comprising (R)-bupropion and dextromethorphan, wherein the dosage form increases a mean Cₘₐₓ of dextromethorphan in a human being on day 1 by at least 60% as compared to that of a reference dosage form on day 1, when orally administered to the human being daily for at least 8 consecutive days, wherein the reference oral dosage form contains the same amount of bupropion as a racemic-bupropion and the same amount of dextromethorphan.

Some embodiments include an oral dosage form comprising (R)-bupropion and dextromethorphan, wherein the dosage form increases a mean Cₘₐₓ of dextromethorphan in a human on day 8 being by at least 20% as compared to that of a reference dosage form on day 8, when orally administered to the human being daily for at least 8 consecutive days, wherein the reference oral dosage form contains the same amount of bupropion as a racemic-bupropion and the same amount of dextromethorphan.

Some embodiments include a method of enhancing the plasma levels of dextromethorphan on day 1 or day 8, comprising orally administering a dosage form containing (R)-bupropion and dextromethorphan, in a much greater extent than orally administering a reference dosage form, when orally administered to a human being daily for at least 8 consecutive days, wherein the reference dosage form contains the same amount of bupropion as a (S)-bupropion and the same amount of dextromethorphan.

Some embodiments include a method of treating a neuropsychiatric disorder, comprising administering a dosage form described in any preceding paragraph to a human being in need thereof.

Some embodiments include a method of treating cold or cough, comprising administering a dosage form described in any preceding paragraph to a human being in need thereof.

Some embodiments include a method of relieving pain, comprising administering a dosage form described in any preceding paragraph to a human being in need thereof.

Some embodiments include a method for treatment of addiction, comprising administering a dosage form described in any preceding paragraph to a human being in need thereof.

Some embodiments include a method of treating a human being comprising orally administering a dosage form containing (S)-bupropion that is at least 95% enantiomerically pure, one or two times per day, to a human being having a condition that is treatable with (S)-bupropion, wherein the amount of (S)-bupropion administered is selected to be about 20% to about 70% of the amount of racemic bupropion that would be administered to treat the same human being for the same condition.

Some embodiments include a method of providing therapeutically effective plasma levels of (R,R)-hydroxybupropion comprising orally administering, one or two times per day, (S)-bupropion that is at least 95% enantiomerically pure, to a human being in need of treatment with (R,R)-hydroxybupropion, wherein (R,R)-hydroxybupropion is at least 97% of the total of amount of (R,R)-hydroxybupropion and (S,S)-hydroxybupropion present in the plasma of the human being, and wherein the method achieves a Cₘₐₓ of (R,R)-hydroxybupropion that is at least about 500 ng/mL in the human being.

Some embodiments include a method of treating a human being comprising orally administering a dosage form containing (S)-bupropion that is at least 95% enantiomerically pure to a human being in need of treatment with (S)-bupropion, wherein the (S)-bupropion is the sole active agent used to treat the human being.

Some embodiments include a method of treating a human being comprising orally administering a dosage form containing about 50 mg to about 100 mg of (S)-bupropion that is at least 95% enantiomerically pure, one or two times per day, to a human being in need of treatment with (S)-bupropion.

Some embodiments include a method of treating a human being comprising orally administering a dosage form containing (S)-bupropion that is at least 95% enantiomerically pure, one or two times per day, to a human being in need of treatment with (S)-bupropion, wherein the method achieves a Cₘₐₓ of (S)-bupropion that is at least about 60 ng/mL.

Some embodiments include a method of treating a human being comprising orally administering a dosage form containing (S)-bupropion that is at least 95% enantiomerically pure, one or two times per day, to a human being in need of treatment with (S)-bupropion, wherein (R,R)-hydroxybupropion is at least 97% of the total amount of (R,R)-hydroxybupropion and (S,S)-hydroxybupropion present in the plasma of the human being.

Some embodiments include a method of providing therapeutically effective plasma levels of (R,R)-hydroxybupropion comprising orally administering, one or two times per day, (S)-bupropion that is at least 95% enantiomerically pure, to a human being in need of treatment with (R,R)-hydroxybupropion, wherein (R,R)-hydroxybupropion is at least 97% of the total of amount of (R,R)-hydroxybupropion and (S,S)-hydroxybupropion present in the plasma of the human being.

Some embodiments include a method of providing therapeutically effective plasma levels of (R,R)-hydroxybupropion comprising orally administering, one or two times per day, (S)-bupropion that is at least 95% enantiomerically pure, to a human being in need of treatment with (R,R)-hydroxybupropion, wherein the method achieves a Cₘₐₓ of (R,R)-hydroxybupropion that is at least about 500 ng/mL in the human being.

Some embodiments include a method of treating a human being comprising orally administering a dosage form containing (S)-bupropion that is at least 95% enantiomerically pure, one or two times per day, to a human being in need of treatment with (S)-bupropion, wherein the method achieves a Cₘₐₓ of (S)-bupropion that is at least about 70 ng/mL.

Some embodiments include a method of treating a human being comprising orally administering a dosage form containing (S)-bupropion that is at least 95% enantiomerically pure, one or two times per day, to a human being in need of treatment with (S)-bupropion, wherein the method achieves an AUC₀₋₁₂of (S)-bupropion that is at least about 600 ng·h/mL.

Some embodiments include a method of treating a human being comprising orally administering a dosage form containing (S)-bupropion that is at least 95% enantiomerically pure, one or two times per day, to a human being in need of treatment with (S)-bupropion, wherein the method achieves a Cₘₐₓ of (R,R)-hydroxybupropion that is at least about 800 ng/mL.

Some embodiments include a method of treating a human being comprising orally administering a dosage form containing (S)-bupropion that is at least 95% enantiomerically pure, one or two times per day, to a human being in need of treatment with (S)-bupropion, wherein the method achieves an AUC₀₋₁₂of (R,R)-hydroxybupropion that is at least about 8,000 ng·h/mL.

Some embodiments include a method of treating a human being comprising orally administering a dosage form containing (S)-bupropion that is at least 95% enantiomerically pure, one or two times per day, to a human being in need of treatment with (S)-bupropion, wherein the method achieves a Cₘₐₓ of erythrohydroxybupropion that is at least about 90 ng/mL.

Some embodiments include a method of treating a human being comprising orally administering a dosage form containing (S)-bupropion that is at least 95% enantiomerically pure, one or two times per day, to a human being in need of treatment with (S)-bupropion, wherein the method achieves an AUC₀₋₁₂of erythrohydroxybupropion that is at least about 1,000 ng·h/mL.

Some embodiments include a method of treating a human being comprising orally administering a dosage form containing (S)-bupropion that is at least 95% enantiomerically pure, one or two times per day, to a human being in need of treatment with (S)-bupropion, wherein the method achieves a Cₘₐₓ of threohydroxybupropion that is at least about 450 ng/mL.

Some embodiments include a method of treating a human being comprising orally administering a dosage form containing (S)-bupropion that is at least 95% enantiomerically pure, one or two times per day, to a human being in need of treatment with (S)-bupropion, wherein the method achieves an AUC₀₋₁₂of threohydroxybupropion that is at least about 5,000 ng·h/mL.

Some embodiments include a dosage form comprising (S)-bupropion which is at least 95% enantiomerically pure.

Some embodiments include a method of enhancing the plasma level of (S)-bupropion or a metabolite thereof, comprising administering any dosage form described herein to a human being in need of treatment with bupropion or a metabolite thereof.

Some embodiments include a method of treating a neurological condition, comprising administering a dosage form described in any preceding paragraph to a human being in need thereof.

Some embodiments include a method of increasing the plasma levels of dextromethorphan comprising administering a combination of (R)-bupropion and dextromethorphan to a human being in need of treatment by dextromethorphan, wherein the (R)-bupropion is at least 95% enantiomerically pure.

Some embodiments include a method of delivering (S)-bupropion to the plasma of a human being comprising: orally administering a dosage form containing (S)-bupropion that is at least 95% enantiomerically pure to the human being.

Some embodiments include a method of delivering both (R)-bupropion and (S)-bupropion to the plasma of a human being comprising: orally administering a dosage form containing (S)-bupropion that is at least 95% enantiomerically pure to the human being, wherein the Cₘₐₓ of (R)-bupropion is within 20% of the Cₘₐₓ of (R)-bupropion that would result from administering the same amount of racemic bupropion to the human being.

Some embodiments include a method of delivering a bupropion to the plasma of a human being comprising: orally administering a dosage form containing (S)-bupropion that is at least 95% enantiomerically pure to the human being, wherein the AUC₀₋₁₂ of (R)-bupropion is within 20% of the AUC₀₋₁₂ of (R)-bupropion that would result from administering the same amount of racemic bupropion to the human being.

Some embodiments include a method of delivering a bupropion to the plasma of a human being comprising: orally administering a dosage form containing (S)-bupropion that is at least 95% enantiomerically pure to the human being, wherein the Cₘₐₓ of (R,R)-hydroxybupropion is within 20% of the Cₘₐₓ of (R,R)-hydroxybupropion that would result from administering the same amount of racemic bupropion to the human being.

### DETAILED DESCRIPTION

Some dosage forms contain an enantiomeric excess of (S)-bupropion, or enantiomerically pure (S)-bupropion. For example, the (S)-bupropion may be at least 70%, at least 80%, at least 85%, at least 90%, at least 95%, at least 96%, at least 97%, at least 98%, at least 99%, at least 99.9%, or at least 99.99% enantiomerically pure, up to (nearly) 100% enantiomeric purity. (S)-bupropion that is 99.99% enantiomerically pure (or enantiomeric excess, or 99.99% ee) contains 99.99% (S)-bupropion and 0.005% [[0.01%]] (R)-bupropion. For convenience, any of the above may be referred to as “(S)-bupropion.” This type of dosage form may be useful in treating conditions where increased levels of (S)-bupropion and/or (R,R)-hydroxybupropion are therapeutically beneficial, or where a human being is in need of treatment with (S)-bupropion and/or (R,R)-hydroxybupropion. Additionally, increased levels of (S)-bupropion or (R,R)-hydroxybupropion may be useful to increase bioavailability or increase plasma levels of dextromethorphan, for example by co-administering the (S)-bupropion with dextromethorphan. Thus, co-administration of an enantiomeric excess of (S)-bupropion, or enantiomerically pure (S)-bupropion, with dextromethorphan, either in the same dosage form or in separate dosage forms, may be useful in treating conditions that respond to dextromethorphan alone, or a combination of (S)-bupropion and dextromethorphan. However, for some treatments, use of dextromethorphan may not be desirable with (S)-bupropion or (R,R)-hydroxybupropion. Thus, some dosage forms are substantially free of dextromethorphan, and some treatments involve administration of (S)-bupropion without co-administration of dextromethorphan.

It has been discovered that orally administering (S)-bupropion is bioequivalent or pharmacokinetically equivalent to administering about twice as much racemic bupropion. For example, a 75 mg dose of (S)-bupropion is bioequivalent to a dose of about 150 mg of racemic bupropion.

Some embodiments include a method of providing a pharmacokinetic equivalent of racemic bupropion to the plasma of a human being, comprising: selecting a human patient in need of a pharmacokinetic profile provided by orally administering a reference dosage form containing a first amount of racemic bupropion at a first dosing frequency; and orally administering a dosage form containing a second amount of (S)-bupropion that is at least 95% enantiomerically pure at the first dosing frequency to achieve the same pharmacokinetic profile that would be achieved by administering the reference dosage form at the first dosing frequency; wherein the first dosing frequency is once daily or twice daily; and wherein the second amount is about 20-70%, about 40-60%, about 45-55%, or about 50% of the first amount. For example, if a particular pharmacokinetic profile is achievable by orally administering a dosage form containing 150 mg of racemic bupropion and the second amount is 40-60% of the first amount, the second amount is 60-90 mg. Thus, in this situation, 60-90 mg of (S)-bupropion would be administered once daily to achieve the same pharmacokinetic profile as would be achieved by administering 150 mg of racemic bupropion once daily; or 60-90 mg of (S)-bupropion would be administered twice daily to achieve the same pharmacokinetic profile as would be achieved by administering 150 mg of racemic bupropion twice daily.

Some embodiments include a method of treating a condition that is treatable with racemic bupropion, comprising: selecting a human patient having the condition that is treatable by orally administering a reference dosage form containing a first amount of racemic bupropion at a first dosing frequency; and orally administering a dosage form containing a second amount of (S)-bupropion that is at least 95% enantiomerically pure at the first dosing frequency to achieve the same therapeutic effect that would be achieved by administering the reference dosage form at the first dosing frequency; wherein the first dosing frequency is once daily or twice daily; and wherein the second amount is about 40-60%, about 45-55%, or about 50% of the first amount. For example, if a condition is treatable by orally administering a dosage form containing 150 mg of racemic bupropion and the second amount is 40-60% of the first amount, the second amount is 60-90 mg. Thus, in this situation, 60-90 mg of (S)-bupropion would be administered once daily to treat a condition so that the same therapeutic effect is achieved as would be achieved by administering 150 mg of racemic bupropion once daily; or 60-90 mg of (S)-bupropion would be administered twice daily to treat a condition so that the same therapeutic effect is achieved as would be achieved by administering 150 mg of racemic bupropion twice daily.

For the purposes of this disclosure, if the dosage form containing enantiomerically pure (S)-bupropion is recognized by the FDA as bioequivalent to a dosage form containing racemic bupropion, then the two dosage forms have the same pharmacokinetic profile.

In some embodiments, the Cₘₐₓof (5)-bupropion, the Cₘₐₓof (R)-bupropion, the combined Cₘₐₓ of (S)-bupropion and (R)-bupropion, the Cₘₐₓ of (R,R)-hydroxybupropion, the Cₘₐₓ of (S,S)-hydroxybupropion, the combined Cₘₐₓ of (R,R)-hydroxybupropion and (S,S)-hydroxybupropion, the Cₘₐₓ of threohydroxybupropion, the Cₘₐₓ of erythrohydroxybupropion, the AUC₀₋₁₂ of (S)-bupropion, the AUC₀₋₁₂ of (R)-bupropion, the combined AUC₀₋₁₂ of (S)-bupropion and (R)-bupropion, the AUC₀₋₁₂ of (R,R)-hydroxybupropion, the AUC₀₋₁₂ of (S,S)-hydroxybupropion, the combined AUC₀₋₁₂ of (R,R)-hydroxybupropion and (S,S)-hydroxybupropion, the AUC₀₋₁₂ of threohydroxybupropion, the AUC₀₋₁₂ of erythrohydroxybupropion, or any combination thereof, are within 20% of each other. If one dosage form has a value of 1, the two dosage forms are deemed to be within 20% of each other if the second dosage form has a value in the range of 0.8-1.2.

Some dosage forms contain an enantiomeric excess of (R)-bupropion, or enantiomerically pure (R)-bupropion. For example, the (R)-bupropion may be at least 70%, at least 80%, at least 85%, at least 90%, at least 95%, at least 96%, at least 97%, at least 98%, at least 99%, at least 99.9%, or at least 99.99% enantiomerically pure, up to (nearly) 100% enantiomeric purity. For convenience, any of the above may be referred to as “(R)-bupropion.” This type of dosage forms may be useful to increase bioavailability or increase plasma levels of dextromethorphan, for example by co-administering the (R)-bupropion with dextromethorphan. Thus, co-administration of an enantiomeric excess of (R)-bupropion, or enantiomerically pure (R)-bupropion, with dextromethorphan, either in a single dosage form or in separate dosage forms, may be useful in treating conditions that respond to dextromethorphan.

The dosage forms and methods described above may be incorporated into methods for reducing an adverse event associated with treatment by bupropion, increasing a bupropion plasma level, such as increasing an (S)-bupropion plasma level, decreasing the dose amount or the number of doses of a bupropion that need to be administered without loss of efficacy, improving a therapeutic property of a bupropion, reducing a trough effect of a bupropion, or other methods.

The dosage forms and methods described above with respect to dextromethorphan may be incorporated into methods for increasing dextromethorphan plasma levels, increasing the metabolic lifetime of dextromethorphan, reducing an adverse event associated with treatment by dextromethorphan, decreasing the dose amount or the number of doses of dextromethorphan that need to be administered without loss of efficacy, decreasing dextrorphan plasma levels, improving a therapeutic property of dextromethorphan, inhibiting the metabolism of dextromethorphan, correcting extensive metabolism of dextromethorphan, improving the antitussive properties of dextromethorphan, treating cough, reducing a trough effect of dextromethorphan, or other methods.

In addition, the dosage forms and methods described above may be applied to neurological disorders, central nervous system disorders, psychiatric disorders, neuropsychiatric disorders, and related conditions.

Administration of enantiomerically enriched (S)-bupropion, or enantiomerically enriched (R)-bupropion, and/or dextromethorphan as described above, or elsewhere in this disclosure, may occur one or more times in a single day, e.g. by oral administration, or for multiple days, such as multiple consecutive days. For example, enantiomerically enriched (S)-bupropion, alone or in combination with dextromethorphan, may be administered once or twice daily for 1, 2, 3, 4, 5, 6, 7, 8, 9-13, 14, 15-20, 21, 22-27, 28, 29, 30, 31, 32-59, 60, 61-89, 90, or more consecutive days. Alternatively, enantiomerically enriched (R)-bupropion, alone or in combination with dextromethorphan, may be administered once or twice daily for 1, 2, 3, 4, 5, 6, 7, 8, 9-13, 14, 15-20, 21, 22-27, 28, 29, 30, 31, 32-59, 60, 61-89, 90, or more consecutive days. The patient may be fasted prior to and/or after oral administration of a dosage form containing (S)-bupropion, or (R)-bupropion, and/or dextromethorphan

In some embodiments, a treatment described above may include administering the dosage form containing (R)-bupropion or (S)-bupropion, alone or in combination with dextromethorphan, once a day for 1, 2, 3, 4, 5, 6, or 7 days, followed by twice a day treatment. For example, the dosage form containing (R)-bupropion or (S)-bupropion could be administered once a day on day 1, 2, and 3, and then twice a day starting on day 4, and continued twice a day for an extended period of time, such as 2, 3, 4, 5, 6, 7, 8, 9-13, 14, 15-20, 21, 22-27, 28, 29, 30, 31, 32-59, 60, 61-89, 90, or more consecutive days, or for the remainder of the treatment period. Starting with a once daily dose for a short period of time, followed by increasing the dose frequency to twice a day, may be helpful in reducing seizure risk.

(S)-Bupropion has the structure shown below.

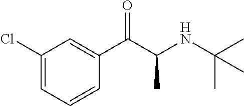 [SMILES: C[C@H](NC(C)(C)C)C(=O)c1cccc(Cl)c1]

Unless otherwise indicated, any reference to a compound herein, such as (S)-bupropion, (R)-bupropion, or dextromethorphan, by structure, name, or any other means, includes pharmaceutically acceptable salts; alternate solid forms, such as polymorphs, crystals, solvates, hydrates, etc.; tautomers; deuterium-modified compounds, such as deuterium modified dextromethorphan; or any chemical species that may rapidly convert to a compound described herein under conditions in which the compounds are used as described herein.

For dosage forms comprising an enantiomeric excess of (S)-bupropion, any suitable amount of (S)-bupropion may be used. In some embodiments, a dosage form contains at least about 40 mg, at least about 50 mg, at least about 60 mg, at least about 70 mg, at least about 80 mg, at least about 90 mg, at least about 100 mg, about 4-90 mg, about 50-100 mg, about 50-150 mg, about 70-95 mg, about 50-70 mg, about 60-80 mg, about 60-90 mg, about 70-80 mg, about 70-74 mg, about 72-76 mg, about 74-76 mg, about 74-78 mg, about 70-90 mg, about 90-110 mg, about 90-120 mg, about 100-200 mg, about 100-140 mg, about 140-160 mg, about 160-200 mg, about 104-106 mg, about 100-110 mg, about 110-120 mg, about 120-130 mg, about 130-140 mg, about 140-150 mg, about 145-155 mg, about 148-152 mg, about 150-200 mg, about 200-400 mg, about 250-350 mg, about 280-320 mg, about 290-310 mg, about 200-250 mg, about 250-300 mg, about 70 mg, about 75 mg, about 80 mg, about 100 mg, about 105 mg, about 110 mg, about 150 mg, about 10-50 mg, about 50-100 mg, about 40-90 mg, about 70-95 mg, or about 50-150 mg, of (S)-bupropion, or any amount in a range bounded by any of these values. Ranges of amounts of (S)-Bu obtained by combining any of the ranges or endpoints above are also contemplated, especially if the range obtained encompasses, or is near, one or more the following values for the amount of (S)-bupropion in the dosage form: about 50 mg, about 60 mg, about 75 mg, or about 90 mg, about 105 mg, or about 150 mg. These are values that are believed to potentially be of particular utility. A dosage form containing an amount of (S)-bupropion listed above may be administered once, twice, or three times a day for a daily dose that is 1, 2, or 3 times that of any dose amount or any dose range listed above, e.g. 2 times 90-120 mg for a daily dose of 180-240 mg, or 3 times 90-120 mg for a daily dose of 270-360 mg. Any daily dose obtained by combining any of the dose ranges or endpoints above and multiplying that result by 1, 2, or 3 are also contemplated, especially if the range obtained encompasses, or is near, one or more the following values: about 150 mg, about 210 mg, or about 250 mg. These are values that are believed to potentially be of particular utility.

Some solid compositions may comprise at least about 5%, at least about 10%, at least about 20%, at least about 50%, at least about 70%, at least about 80%, about 10-30%, about 30-50%, about 50-80%, about 80-95%, about 10-50%, about 30-70%, or about 50-90% of (S)-bupropion by weight.

In some embodiments, the dosage form may be free, or substantially free, of any active pharmaceutical agents, or drugs, other than the (S)-bupropion. For example, the dosage form may contain less than 10% by weight, less than 5% by weight, less than 1% by weight, or less than 0.1% by weight of any other active pharmaceutical agent, as compared to the weight of the (S)-bupropion. In some embodiments, the dosage form may contain less than 10% by weight, less than 5% by weight, less than 1% by weight, or less than 0.1% by weight of dextromethorphan, as compared to the weight of the (S)-bupropion, or may contain no dextromethorphan. Alternatively, (S)-bupropion may be combined with another compound, such as dextromethorphan, in the dosage form.

Administering (S)-bupropion may be useful in increasing plasma levels of (S)-bupropion by at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 4-fold, at least about 5-fold, about 5-20 fold, at least about 10-fold, at least about 20-fold, at least about 50-fold, at least about 100-fold, at least about 150-fold, at least about 200-fold, or more, about 10-15 fold, about 10-25 fold, about 5-20 fold, about 20-30 fold, about 30-40 fold, about 40-50 fold, about 50-60 fold, about 60-70 fold, about 70-80 fold, about 80-90 fold, about 90-100 fold, about 100-110 fold, about 110-120 fold, about 120-130 fold, about 130-140 fold, about 140-150 fold, about 150-160 fold, about 160-170 fold, about 170-180 fold, about 180-190 fold, or about 190-200 fold, as compared to the plasma level of (R)-bupropion obtained by administering a dosage form containing the same amount of (R)-bupropion to the human being.

In some embodiments, the method is effective in increasing the AUC₀₋₁₂, such as the average AUC₀₋₁₂, the mean AUC₀₋₁₂, the median AUC₀₋₁₂, or the AUC₀₋₁₂ of an individual, of (S)-bupropion by at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 4-fold, at least about 5-fold, at least about 8-fold, at least about 10-fold, about 10-fold, at least about 15-fold, at least about 20-fold, at least about 25-fold, at least about 50-fold, at least about 100-fold, at least about 150-fold, at least about 200-fold, or more, about 10-15 fold, about 10-25 fold, about 5-20 fold, about 20-30 fold, about 30-40 fold, about 40-50 fold, about 50-60 fold, about 60-70 fold, about 70-80 fold, about 80-90 fold, about 90-100 fold, about 100-110 fold, about 110-120 fold, about 120-130 fold, about 130-140 fold, about 140-150 fold, about 150-160 fold, about 160-170 fold, about 170-180 fold, about 180-190 fold, or about 190-200 fold, as compared to the AUC₀₋₁₂ of (R)-bupropion obtained administering a dosage form containing the same amount of (R)-bupropion to the human being.

In some embodiments, the method achieves an AUC₀₋₁₂, such as the average AUC₀₋₁₂, the mean AUC₀₋₁₂, the median AUC₀₋₁₂, or the AUC₀₋₁₂ of an individual, of (S)-bupropion, or of (S)-bupropion and (R)-bupropion combined, that is at least about 300 ng·hr/mL, at least about 400 ng·hr/mL, at least about 500 ng·hr/mL, at least about 600 ng·hr/mL, at least about 700 ng·hr/mL, at least about 750 ng·hr/mL, at least about 800 ng·hr/mL, at least about 850 ng·hr/mL, at least about 900 ng·hr/mL, at least about 950 ng·hr/mL, at least about 1,000 ng·hr/mL, at least about 1,100 ng·hr/mL, at least about 1,200 ng·hr/mL, up to about 1,200 ng·hr/mL, up to about 1,300 ng·hr/mL, up to about 1,400 ng·hr/mL, up to about 1,500 ng·hr/mL, up to about 1,600 ng·hr/mL, up to about 1,700 ng·hr/mL, up to about 1,800 ng·hr/mL, about 300-400 ng·hr/mL, about 400-500 ng·hr/mL, about 500-600 ng·hr/mL, about 600-700 ng·hr/mL, about 700-800 ng·r/mL, about 800-900 ng·hr/mL, about 900-1,000 ng·hr/mL, about 1,000-1,100 ng·hr/mL, about 1,100-1,200 ng·hr/mL, about 1,200-1,300 ng·hr/mL, about 1,300-1,400 ng·hr/mL, about 1,400-1,500 ng·hr/mL, about 1,500-1,600 ng·hr/mL, about 1,600-1,700 ng·hr/mL, about 1,700-1,800 ng·hr/mL, about 300-600 ng·hr/mL, about 600-900 ng·hr/mL, about 900-1,200 ng·hr/mL, about 1,200-1,500 ng·hr/mL, about 1,500-1,800 ng·hr/mL, about 300-800 ng·hr/mL, about 800-1,300 ng·hr/mL, about 1,300-1,800 ng·hr/mL, or about 300-1,800 ng·hr/mL. Ranges of AUC₀₋₁₂ obtained by combining any of the ranges or endpoints above are also contemplated, especially if the range obtained encompasses, or is near, one or more the following values for AUC₀₋₁₂: 350 ng·hr/mL, 400 ng·hr/mL, 750 ng·hr/mL, 900 ng·hr/mL, 1,150 ng·hr/mL, or 1,400 ng·hr/mL. These are values that are believed to potentially be of particular utility. The AUC ranges targeted may be observed on day 1, day 2, day 3, day 4, day 5, day 6, day 7, day 8, or later.

In some embodiments, the method is effective in increasing the Cₘₐₓ, such as the average Cₘₐₓ, the mean Cₘₐₓ, the median Cₘₐₓ, or the Cₘₐₓ of an individual, of (S)-bupropion by at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 4-fold, at least about 5-fold, at least about 8-fold, about 5-20 fold, at least about 10-fold, about 10-fold, at least about 20-fold, at least about 25-fold, at least about 50-fold, at least about 100-fold, at least about 150-fold, at least about 200-fold, or more, about 10-15 fold, about 10-25 fold, about 5-20 fold, about 20-30 fold, about 30-40 fold, about 40-50 fold, about 50-60 fold, about 70-80 fold, about 80-90 fold, about 90-100 fold, about 100-110 fold, about 110-120 fold, about 120-130 fold, about 130-140 fold, about 140-150 fold, about 150-160 fold, about 160-170 fold, about 170-180 fold, about 180-190 fold, or about 190-200 fold, as compared to administering a dosage form containing the same amount of (R)-bupropion to the human being.

In some embodiments, the method achieves a Cₘₐₓ, such as the average Cₘₐₓ, the mean Cₘₐₓ, the median Cₘₐₓ, or the Cₘₐₓ of an individual, of (S)-bupropion, or a combined Cₘₐₓ of (S)-bupropion and (R)-bupropion, that is at least about 30 ng/mL, at least about 50 ng/mL, at least about 60 ng/mL, at least about 70 ng/mL, at least about 80 ng/mL, at least about 90 ng/mL, at least about 95 ng/mL, at least about 100 ng/mL, at least about 105 ng/mL, at least about 110 ng/mL, at least about 115 ng/mL, at least about 120 ng/mL, at least about 125 ng/mL, at least about 130 ng/mL, at least about 140 ng/mL, up to about 130 ng/mL, up to about 140 ng/mL, up to about 150 ng/mL, up to about 160 ng/mL, up to about 170 ng/mL, up to about 180 ng/mL, up to about 190 ng/mL, up to about 200 ng/mL, up to about 210 ng/mL, up to about 220 ng/mL, about 30-40 ng/mL, about 40-50 ng/mL, about 50-60 ng/mL, about 60-70 ng/mL, about 70-80 ng/mL, about 80-90 ng/mL, about 90-100 ng/mL, about 100-110 ng/mL, about 110-120 ng/mL, about 120-130 ng/mL, about 130-140 ng/mL, about 140-150 ng/mL, about 150-160 ng/mL, about 160-170 ng/mL, about 170-180 ng/mL, about 180-190 ng/mL, about 190-200 ng/mL, about 200-210 ng/mL, about 210-220 ng/mL, about 50-70 ng/mL, about 70-90 ng/mL, about 30-60 ng/mL, about 60-90 ng/mL, about 90-120 ng/mL, about 120-160 ng/mL, about 160-220 ng/mL, about 30-90 ng/mL, about 90-150 ng/mL, about 150-220 ng/mL, about 30-110 ng/mL, about 130-190 ng/mL, about 110-220 ng/mL, or about 30-220 ng/mL. Ranges of Cₘₐₓ obtained by combining any of the ranges or endpoints above are also contemplated, especially if the range obtained encompasses, or is near, one or more the following values for Cₘₐₓ: 45 ng/mL, 90 ng/mL, 130 ng/mL, 160 ng/mL, or 190 ng/mL. These are values that are believed to potentially be of particular utility. The Cₘₐₓ ranges targeted may be observed on day 1, day 2, day 3, day 4, day 5, day 6, day 7, day 8, or later.

In some embodiments, the method is effective in increasing the Cₘᵢₙ, such as the average Cₘᵢₙ, the mean Cₘᵢₙ, the median Cₘᵢₙ, or the Cₘᵢₙ of an individual, of (S)-bupropion by at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 4-fold, at least about 5-fold, at least about 8-fold, at least about 10-fold, at least about 20-fold, at least about 50-fold, at least about 100-fold, at least about 150-fold, at least about 200-fold, or more, about 10-15 fold, about 10-25 fold, about 5-20 fold, about 20-30 fold, about 30-40 fold, about 40-50 fold, about 50-60 fold, about 70-80 fold, about 80-90 fold, about 90-100 fold, about 100-110 fold, about 110-120 fold, about 120-130 fold, about 130-140 fold, about 140-150 fold, about 150-160 fold, about 160-170 fold, about 170-180 fold, about 180-190 fold, or about 190-200 fold, as compared to administering a dosage form containing the same amount of (R)-bupropion to the human being.

In some embodiments, the method achieves a Cₘᵢₙ, such as the average Cₘᵢₙ, the mean Cₘᵢₙ, the median Cₘᵢₙ, or the Cₘᵢₙ of an individual, of (S)-bupropion that is at least about 20 ng/mL, at least about 25 ng/mL, at least about 30 ng/mL, at least about 35 ng/mL, at least about 40 ng/mL, about 20-60 ng/mL, about 25-30 ng/mL, about 30-35 ng/mL, about 35-40 ng/mL, about 30-40 ng/mL, about 40-45 ng/mL, about 45-50 ng/mL, about 30-50 ng/mL, about 40-50 ng/mL, or about 50-60 ng/mL. Ranges of Cₘᵢₙ obtained by combining any of the ranges or endpoints above are also contemplated, especially if the range obtained encompasses, or is near, one or more the following values for Cₘᵢₙ: 20 ng/mL, 30 ng/mL, 40 ng/mL, or 50 ng/mL. These are values that are believed to potentially be of particular utility.

Administering (S)-bupropion may be useful in increasing plasma levels of (R,R)-hydroxybupropion, by at least about 1.1-fold, at least about 1.5-fold, at least about 2-fold, at least about 3-fold, at least about 5-fold, at least about 10-fold, at least about 15-fold, at least about 20-fold, at least about 40-fold, at least about 50-fold, at least about 100-fold, at least about 150-fold, at least about 200-fold, or more, about 5-10 fold, about 5-25 fold, about 5-20 fold, about 20-30 fold, about 30-40 fold, about 40-50 fold, about 50-60 fold, about 70-80 fold, about 80-90 fold, about 90-100 fold, about 100-110 fold, about 110-120 fold, about 120-130 fold, about 130-140 fold, about 140-150 fold, about 150-160 fold, about 160-170 fold, about 170-180 fold, about 180-190 fold, or about 190-200 fold, as compared to administering a dosage form containing the same amount of (R)-bupropion to the human being.

Administering (S)-bupropion may be useful in increasing the AUC₀₋₁₂, such as the average AUC₀₋₁₂, the mean AUC₀₋₁₂, the median AUC₀₋₁₂, or the AUC₀₋₁₂ of an individual, of (R,R)-hydroxybupropion, by at least about 1.1-fold, at least about 1.5-fold, at least about 2-fold, at least about 3-fold, at least about 5-fold, about 6-fold, at least about 10-fold, at least about 15-fold, at least about 20-fold, at least about 40-fold, at least about 50-fold, at least about 100-fold, at least about 150-fold, at least about 200-fold, or more, about 5-10 fold, about 5-25 fold, about 5-20 fold, about 20-30 fold, about 30-40 fold, about 40-50 fold, about 50-60 fold, about 70-80 fold, about 80-90 fold, about 90-100 fold, about 100-110 fold, about 110-120 fold, about 120-130 fold, about 130-140 fold, about 140-150 fold, about 150-160 fold, about 160-170 fold, about 170-180 fold, about 180-190 fold, or about 190-200 fold, as compared to administering a dosage form containing the same amount of (R)-bupropion to the human being. In some embodiments, the method achieves an AUC₀₋₁₂, such as the average AUC₀₋₁₂, the mean AUC₀₋₁₂, the median AUC₀₋₁₂, or the AUC₀₋₁₂ of an individual, of (R,R)-hydroxybupropion, or (5,5)-hydroxybupropion and (R,R)-hydroxybupropion combined, that is at least about 1,000 ng·hr/mL, at least about 3,000 ng·hr/mL, at least about 4,000 ng·hr/mL, at least about 6,000 ng·hr/mL, at least about 7,000 ng·hr/mL, at least about 7,500 ng·hr/mL, at least about 8,000 ng·hr/mL, at least about 8,500 ng·hr/mL, at least about 9,000 ng·hr/mL, at least about 9,500 ng·hr/mL, at least about 10,000 ng·hr/mL, at least about 12,000 ng·hr/mL, at least about 13,000 ng·hr/mL, about 4,000-6,000 ng·hr/mL, about 6,000-8,000 ng·hr/mL, about 8,000-10,000 ng·hr/mL, about 10,000-12,000 ng·hr/mL, about 12,000-14,000 ng·hr/mL, about 14,000-16,000 ng·hr/mL, about 16,000-18,000 ng·hr/mL, about 18,000-20,000 ng·hr/mL, about 20,000-22,000 ng·hr/mL, about 22,000-24,000 ng·hr/mL, about 4,000-10,000 ng·hr/mL, about 10,000-16,000 ng·hr/mL, about 16,000-24,000 ng·hr/mL, about 4,000-24,000 ng·hr/mL, up to about 14,000 ng·hr/mL, up to about 16,000 ng·hr/mL, up to about 18,000 ng·hr/mL, up to about 20,000 ng·hr/mL, up to about 22,000 ng·hr/mL, up to about 24,000 ng·hr/mL, or up to about 30,000 ng·hr/mL. Ranges of AUC₀₋₁₂ obtained by combining any of the ranges or endpoints above are also contemplated, especially if the range obtained encompasses, or is near, one or more the following values for AUC₀₋₁₂: 4,000 ng·hr/mL, 5,000 ng/mL, 10,000 ng·hr/mL, 13,400 ng·hr/mL, 16,000 ng·hr/mL, or 22,000 ng·hr/mL. These are values that are believed to potentially be of particular utility. The AUC ranges targeted may be observed on day 1, day 2, day 3, day 4, day 5, day 6, day 7, day 8, or later.

Administering (S)-bupropion may be useful in increasing the Cₘₐₓ, such as the average Cₘₐₓ, the mean Cₘₐₓ, the median Cₘₐₓ, or the Cₘₐₓ of an individual, of (R,R)-hydroxybupropion, by at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 4-fold, at least about 5-fold, about 6-fold, at least about 10-fold, at least about 20-fold, at least about 50-fold, at least about 100-fold, at least about 150-fold, at least about 200-fold, or more, about 5-10 fold, about 10-20 fold, about 5-20 fold, about 20-30 fold, about 30-40 fold, about 40-50 fold, about 50-60 fold, about 70-80 fold, about 80-90 fold, about 90-100 fold, about 100-110 fold, about 110-120 fold, about 120-130 fold, about 130-140 fold, about 140-150 fold, about 150-160 fold, about 160-170 fold, about 170-180 fold, about 180-190 fold, or about 190-200 fold, as compared to administering a dosage form containing the same amount of (R)-bupropion to the human being.

In some embodiments, the method achieves a Cₘₐₓ, such as the average Cₘₐₓ, the mean Cₘₐₓ, the median Cₘₐₓ, or the Cₘₐₓ of an individual, of (R,R)-hydroxybupropion or (S,S)-hydroxybupropion and (R,R)-hydroxybupropion combined, that is at least about 90 ng/mL, at least about 150 ng/mL, at least about 200 at least about 300 ng/mL, at least about 400 ng/mL, at least about 500 ng/mL, at least about 600 ng/mL, at least about 700 ng/mL, at least about 800 ng/mL, at least about 900 ng/mL, at least about 1,000 ng/mL, at least about 1,100 ng/mL, at least about 1,200 ng/mL, at least about 1,300 ng/mL, up to about 1,400 ng/mL, up to about 1,500 ng/mL, up to about 1,600 ng/mL, up to about 1,700 ng/mL, up to about 1,800 ng/mL, up to about 1,900 ng/mL, up to about 2,000 ng/mL, up to about 2,100 ng/mL, up to about 2,200 ng/mL, up to about 2,300 ng/mL, about 300-400 ng/mL, about 400-500 ng/mL, about 500-600 ng/mL, about 600-700 ng/mL, about 700-800 ng/mL, about 800-900 ng/mL, about 900-1,000 ng/mL, about 1,000-1,100 ng/mL, about 1,100-1,200 ng/mL, about 1,200-1,300 ng/mL, about 1,300-1,400 ng/mL, about 1,400-1,500 ng/mL, about 1,500-1,600 ng/mL, about 1,600-1,700 ng/mL, about 1,700-1,800 ng/mL, about 1,800-1,900 ng/mL, about 1,900-2,000 ng/mL, about 2,000-2,100 ng/mL, about 2,100-2,200 ng/mL, about 2,200-2,300 ng/mL, about 300-800 ng/mL, about 800-1,300 ng/mL, about 1,300-2,300 ng/mL, or about 300-2,300 ng/mL. Ranges of Cₘₐₓ obtained by combining any of the ranges or endpoints above are also contemplated, especially if the range obtained encompasses, or is near, one or more of the following values for Cₘₐₓ: 400 ng/mL, 950 ng/mL, 1,100 ng/mL, 1,250 ng/mL, 1,400 ng/mL, or 2,100 ng/mL. These are values that are believed to potentially be of particular utility. The Cₘₐₓ ranges targeted may be observed on day 1, day 2, day 3, day 4, day 5, day 6, day 7, day 8, or later.

Administering (S)-bupropion may be useful in increasing the Cₘᵢₙ, such as the average Cₘᵢₙ, the mean Cₘᵢₙ, the median Cₘᵢₙ, or the Cₘᵢₙ of an individual, of (R,R)-hydroxybupropion, by at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 4-fold, at least about 5-fold, at least about 10-fold, at least about 20-fold, at least about 50-fold, at least about 60-fold, at least about 100-fold, at least about 150-fold, at least about 200-fold, or more, about 1-5 fold, about 5-10 fold, about 5-20 fold, about 20-30 fold, about 30-40 fold, about 40-50 fold, about 50-60 fold, about 60-70 fold, about 70-80 fold, about 80-90 fold, about 90-100 fold, about 100-110 fold, about 110-120 fold, about 120-130 fold, about 130-140 fold, about 140-150 fold, about 150-160 fold, about 160-170 fold, about 170-180 fold, about 180-190 fold, or about 190-200 fold, as compared to administering a dosage form containing the same amount of (R)-bupropion to the human being.

In some embodiments, the method achieves a Cₘᵢₙ, such as the average Cₘᵢₙ, the mean Cₘᵢₙ, the median Cₘᵢₙ, or the Cₘᵢₙ of an individual, of (R,R)-hydroxybupropion that is at least about 150 ng/mL, at least about 200 ng/mL, at least about 300 ng/mL, at least about 400 ng/mL, at least about 500 ng/mL, at least about 600 ng/mL, at least about 700 ng/mL, at least about 800 ng/mL, at least about 900 ng/mL, at least about 1,000 ng/mL, at least about 1,100 ng/mL, at least about 1,200 ng/mL, up to about 1,300 ng/mL, up to about 1,400 ng/mL, up to about 1,500 ng/mL, up to about 1,600 ng/mL, up to about 1,700 ng/mL, up to about 1,800 ng/mL, up to about 1,900 ng/mL, up to about 2,000 ng/mL, up to about 2,100 ng/mL, up to about 2,200 ng/mL, up to about 2,300 ng/mL, about 200-300 ng/mL, about 300-400 ng/mL, about 400-500 ng/mL, about 500-600 ng/mL, about 600-700 ng/mL, about 700-800 ng/mL, about 800-900 ng/mL, about 900-1,000 ng/mL, about 1,000-1,100 ng/mL, about 1,100-1,200 ng/mL, about 1,200-1,300 ng/mL, about 1,300-1,400 ng/mL, about 1,400-1,500 ng/mL, about 1,500-1,600 ng/mL, about 300-700 ng/mL, about 700-1,100 ng/mL, about 1,100-1,600 ng/mL, or about 800-1,200 ng/mL. Ranges of Cₘᵢₙ obtained by combining any of the ranges or endpoints above are also contemplated, especially if the range obtained encompasses, or is near, one or more of the following values for Cₘᵢₙ: 350 ng/mL, 600 ng/mL, 800 ng/mL, 1,000 ng/mL, 1,200 ng/mL, or 1,400 ng/mL. These are values that are believed to potentially be of particular utility.

Administering (S)-bupropion to a human being may result in (R,R)-hydroxybupropion being at least 90%, at least 95%, at least 97%, at least 97.2%, at least at least 97.4%, at least 97.6%, at least 97.8%, or at least 98% of the total of amount of (R,R)-hydroxybupropion and (S,S)-hydroxybupropion present in the plasma of the human being.

In some embodiments, the method achieves an AUC₀₋₁₂, such as the average AUC₀₋₁₂, the mean AUC₀₋₁₂, the median AUC₀₋₁₂, or the AUC₀₋₁₂ of an individual, of erythrohydroxybupropion that is at least about 500 ng·hr/mL, at least about 600 ng·hr/mL, at least about 800 ng·hr/mL, at least about 1,000 ng·hr/mL, at least about 1,200 ng·hr/mL, at least about 1,400 ng·hr/mL, at least about 1,500 ng·hr/mL, at least about 1,600 ng·hr/mL, at least about 1,800 ng·hr/mL, at least about 2,000 ng·hr/mL, up to about 1,400 ng·hr/mL, up to about 1,600 ng·hr/mL, up to about 1,800 ng·hr/mL, up to about 2,000 ng·hr/mL, up to about 2,200 ng·hr/mL, up to about 2,400 ng·hr/mL, up to about 2,600 ng·hr/mL, up to about 2,800 ng·hr/mL, up to about 3,000 ng·hr/mL, about 500-600 ng·hr/mL, about 600-800 ng·hr/mL, about 800-1,000 ng·hr/mL, about 1,000-1,200 ng·hr/mL, about 1,200-1,400 ng·hr/mL, about 1,400-1,600 ng·hr/mL, about 1,600-1,800 ng·hr/mL, about 1,800-2,000 ng·hr/mL, about 2,000-2,200 ng·hr/mL, about 2,200-2,400 ng·hr/mL, about 2,400-2,600 ng·hr/mL, about 2,600-2,800 ng·hr/mL, about 2,800-3,000 ng·hr/mL, about 500-1,000 ng·hr/mL, about 1,000-1,500 ng·hr/mL, about 1,500-2,000 ng·hr/mL, about 2,000-2,500 ng·hr/mL, about 2,500-3,000 ng·hr/mL, about 500-1,500 ng·hr/mL, about 1,500-3,000 ng·hr/mL, about 2,000-3,000 ng·hr/mL, or about 500-3,000 ng·hr/mL. Ranges of AUC₀₋₁₂ obtained by combining any of the ranges or endpoints above are also contemplated, especially if the range obtained encompasses, or is near, one or more the following values for AUC₀₋₁₂: 500 ng·hr/mL, 1,300 ng·hr/mL, 1,500 ng·hr/mL, 1,800 ng·hr/mL, or 2,600 ng·hr/mL. These are values that are believed to potentially be of particular utility. The AUC ranges targeted may be observed on day 1, day 2, day 3, day 4, day 5, day 6, day 7, day 8, or later.

In some embodiments, the method achieves a Cₘₐₓ, such as the average Cₘₐₓ, the mean Cₘₐₓ, the median Cₘₐₓ, or the Cₘₐₓ of an individual, of erythrohydroxybupropion that is at least about 40 ng/mL, at least about 60 ng/mL, at least about 80 ng/mL, at least about 90 ng/mL, at least about 100 ng/mL, at least about 120 ng/mL, at least about 140 ng/mL, at least about 160 ng/mL, at least about 180 ng/mL, at least about 200 ng/mL, up to about 160 ng/mL, up to about 180 ng/mL, up to about 200 ng/mL, up to about 220 ng/mL, up to about 240 ng/mL, up to about 260 ng/mL, up to about 280 ng/mL, about 40-60 ng/mL, about 60-80 ng/mL, about 80-100 ng/mL, about 100-120 ng/mL, about 120-140 ng/mL, about 140-160 ng/mL, about 160-180 ng/mL, about 180-200 ng/mL, about 200-220 ng/mL, about 220-240 ng/mL, about 240-260 ng/mL, about 260-280 ng/mL, about 280-300 ng/mL, about 40-100 ng/mL, about 100-150 ng/mL, about 150-200 ng/mL, about 200-250 ng/mL, about 250-280 ng/mL, about 40_{—1}20 ng/mL, about 120-200 ng/mL, about 200-280 ng/mL, or about 40-280 ng/mL. Ranges of Cₘₐₓ obtained by combining any of the ranges or endpoints above are also contemplated, especially if the range obtained encompasses, or is near, one or more the following values for Cₘₐₓ: 60 ng/mL, 120 ng/mL, 130 ng/mL, 200 ng/mL, or 240 ng/mL. These are the values that are believed to potentially be of particular utility. The Cₘₐₓ ranges targeted may be observed on day 1, day 2, day 3, day 4, day 5, day 6, day 7, day 8, or later.

In some embodiments, the method achieves an AUC₀₋₁₂, such as the average AUC₀₋₁₂, the mean AUC₀₋₁₂, the median AUC₀₋₁₂, or the AUC₀₋₁₂ of an individual, of threohydroxybupropion that is at least 1,000 ng·hr/mL, at least about 2,000 ng·hr/mL, at least about 3,000 ng·hr/mL, at least about 4,000 ng·hr/mL, at least about 5,000 ng·hr/mL, at least about 6,000 ng·hr/mL, at least about 7,000 ng·hr/mL, at least about 8,000 ng·hr/mL, up to about 8,000 ng·hr/mL, up to about 9,000 ng·hr/mL, up to about 10,000 ng·hr/mL, up to about 11,000 ng·hr/mL, up to about 12,000 ng·hr/mL, up to about 13,000 ng·hr/mL, up to about 14,000 ng·hr/mL, up to about 15,000 ng·hr/mL, about 2,000-15,000 ng·hr/mL, about 2,000-3,000 ng·hr/mL, about 3,000-4,000 ng·hr/mL, about 4,000-5,000 ng·hr/mL, about 5,000-6,000 ng·hr/mL, about 6,000-7,000 ng·hr/mL, about 7,000-8,000 ng·hr/mL, about 8,000-9,000 ng·hr/mL, about 9,000-10,000 ng·hr/mL, about 10,000-11,000 ng·hr/mL, about 11,000-12,000 ng·hr/mL, about 12,000-13,000 ng·hr/mL, about 13,000-14,000 ng·hr/mL, about 14,000-15,000 ng·hr/mL, about 2,000-6,000 ng·hr/mL, about 6,000-10,000 ng·hr/mL, about 10,000-15,000 ng·hr/mL, about 2,000-9,000 ng·hr/mL, or about 9,000-15,000 ng·hr/mL. Ranges of AUC₀₋₁₂ obtained by combining any of the ranges or endpoints above are also contemplated, especially if the range obtained encompasses, or is near, one or more the following values for AUC₀₋₁₂: 3,000 ng·hr/mL, 6,000 ng·hr/mL, 8,000 ng·hr/mL, or 12,000 ng·hr/mL. These are the values that are believed to potentially be of particular utility. The AUC ranges targeted may be observed on day 1, day 2, day 3, day 4, day 5, day 6, day 7, day 8, or later.

In some embodiments, the method achieves a Cₘₐₓ, such as the average Cₘₐₓ, the mean Cₘₐₓ, the median Cₘₐₓ, or the Cₘₐₓ of an individual, of threohydroxybupropion that is at least about 200 ng/mL, at least about 300 ng/mL, at least about 400 ng/mL, at least about 450 ng/mL, at least about 500 ng/mL, at least about 600 ng/mL, at least about 700 ng/mL, at least about 800 ng/mL, up to about 800 ng/mL, up to about 900 ng/mL, up to about 1,000 ng/mL, up to about 1,100 ng/mL, up to about 1,200 ng/mL, up to about 1,300 ng/mL, up to about 1,400 ng/mL, about 200-300 ng/mL, about 300-400 ng/mL, about 400-500 ng/mL, about 500-600 ng/mL, about 600-700 ng/mL, about 700-800 ng/mL, about 800-900 ng/mL, about 900-1000 ng/mL, about 1,000-1,100 ng/mL, about 1,100-1,200 ng/mL, about 1,200-1,300 ng/mL, about 1,300-1,400 ng/mL, about 200-500 ng/mL, about 500-800 ng/mL, about 800-1,100 ng/mL, about 1,100-1,400 ng/mL, about 200-800 ng/mL, about 800-1400 ng/mL, 400-600 ng/mL, or about 200-1,400 ng/mL. Ranges of Cₘₐₓ obtained by combining any of the ranges or endpoints above are also contemplated, especially if the range obtained encompasses, or is near, one or more the following values for Cₘₐₓ: 300 ng/mL, 500 ng/mL, 600 ng/mL, 900 ng/mL, or 1,200 ng/mL. These are values that are believed to potentially be of particular utility. The Cₘₐₓ ranges targeted may be observed on day 1, day 2, day 3, day 4, day 5, day 6, day 7, day 8, or later.

(R)-Bupropion has the structure shown below.

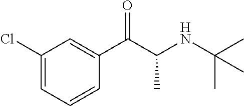 [SMILES: C[C@@H](NC(C)(C)C)C(=O)c1cccc(Cl)c1]

For dosage forms comprising an enantiomeric excess of (R)-bupropion, any suitable amount of (R)-bupropion may be used. In some embodiments, a dosage form contains at least about 80 mg, at least about 90 mg, at least about 100 mg, about 90-110 mg, about 100-200 mg, about 100-150 mg, about 100-140 mg, about 140-160 mg, about 160-200 mg, about 104-106 mg, about 100-110 mg, about 110-120 mg, about 120-130 mg, about 130-140 mg, about 140-150 mg, about 145-155 mg, about 148-152 mg, about 150-200 mg, about 200-400 mg, about 250-350 mg, about 280-320 mg, about 290-310 mg, about 200-250 mg, about 250-300 mg, about 100 mg, about 105 mg, about 110 mg, or about 150 mg of (R)-bupropion, or any amount in a range bounded by any of these values. Such a dosage form may be administered with dextromethorphan, wherein the dextromethorphan may be administered in a separate dosage form, or in the same dosage form as the (R)-bupropion. A dosage form containing an amount of (R)-bupropion listed above may be administered once, twice, or three times a day for a daily dose amount that is 1, 2, or 3 times that of any dose amount or dose range listed above.

In some embodiments, the dosage form may be free, or substantially free, of any active pharmaceutical agents, or drugs, other than the (R)-bupropion and/or dextromethorphan. For example, the dosage form may contain less than 10% by weight, less than 5% by weight, less than 1% by weight, or less than 0.1% by weight of any other active pharmaceutical agent, as compared to the weight of the (R)-bupropion plus dextromethorphan.

Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 60% (w/w) to about 70% (w/w), about 70% (w/w) to about 80% (w/w), or about 80% (w/w) to about 90% (w/w) of (R)-bupropion. As explained above, depending upon the particular therapeutic need, a dosage form containing an enantiomeric excess of (S)-bupropion or an enantiomeric excess of (R)-bupropion, may be administered with dextromethorphan, or may contain dextromethorphan.

Dextromethorphan has the structure shown below.

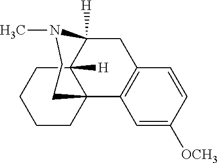 [SMILES: COc1ccc2c(c1)[C@]13CCCC[C@@H]1[C@H](C2)N(C)CC3]

Unless otherwise indicated, any reference to a compound herein, such as (S)-bupropion, (R)-bupropion, and/or dextromethorphan, by structure, name, or any other means, includes pharmaceutically acceptable salts; alternate solid forms, such as polymorphs, crystals, solvates, hydrates, etc.; tautomers; deuterium-modified compounds, such as deuterium modified dextromethorphan; or any chemical species that may rapidly convert to a compound described herein under conditions in which the compounds are used as described herein.

The dextromethorphan may be deuterium enriched, or may have natural isotopic abundance.

Dextromethorphan is used as a cough suppressant. According to the FDA's dextromethorphan product labeling requirement under the OTC Monograph [21CFR341.74], dextromethorphan should be dosed 6 times a day (every 4 hours), 4 times a day (every 6 hours), or 3 times a day (every 8 hours).

Dextromethorphan is rapidly metabolized in the human liver. This rapid hepatic metabolism may limit systemic drug exposure in individuals who are extensive metabolizers. Human beings can be: 1) extensive metabolizers of dextromethorphan those who rapidly metabolize dextromethorphan; 2) poor metabolizers of dextromethorphan those who only poorly metabolize dextromethorphan; or 3) intermediate metabolizers of dextromethorphan those whose metabolism of dextromethorphan is somewhere between that of an extensive metabolizer and a poor metabolizer. Extensive metabolizers can also be ultra-rapid metabolizers. Extensive metabolizers of dextromethorphan represent a significant portion of the human population. Dextromethorphan (DM) can, for example, be metabolized to dextrorphan (DX). The DM/DX ratio can often be used to represent the extent of metabolism of dextromethorphan. A lower DM/DX ratio represents higher extent of metabolism of dextromethorphan. A person having a DM/DX ratio of <0.3 is usually considered a phenotypically extensive metabolizer.

When given the same oral dose of dextromethorphan, plasma levels of dextromethorphan are significantly higher in poor metabolizers or intermediate metabolizers as compared to extensive metabolizers of dextromethorphan. The low plasma concentrations of dextromethorphan can limit its clinical utility as a single agent for extensive metabolizers and possibly intermediate metabolizers of dextromethorphan. Some therapeutically active compounds, including antidepressants such as (S)-bupropion or (R)-bupropion, inhibit the metabolism of dextromethorphan, and raise the plasma concentration of dextromethorphan, and can thus improve its therapeutic efficacy. Similarly, (S)-bupropion or (R)-bupropion may allow dextromethorphan to be given less often, such as once a day instead of twice a day, once a day instead of three times a day, once a day instead of four times a day, twice a day instead of three times a day, or twice a day instead of four times a day, without loss of therapeutic efficacy. Also (S)-bupropion or (R)-bupropion may allow dextromethorphan to be given less amount of each dose or total dose or both.

A dosage form containing dextromethorphan used to this end, either administered as an adjunctive dosage form, or combined into a single dosage form with the (S)-bupropion or the (R)-bupropion, may contain any suitable amount of dextromethorphan, such as about 1-150 mg, about 10-100 mg, about 10-50 mg, about 20-50 mg, about 10-20, about 15-25, about 20-30, about 25-35, about 30-40, about 35-45, about 30-50 mg, about 40-50 mg, about 43-48 mg, about 44-46 mg, about 50-100 mg, about 50-80 mg, about 80-100 mg about 85-95 mg, about 88-92 mg, about 100-150 mg, about 30 mg, about 45 mg, or about 60 mg of dextromethorphan, or any amount of dextromethorphan in a range bounded by any of these values. A dosage form containing an amount of dextromethorphan listed above may be administered once, twice, or three times a day for a daily dose that is 1, 2, or 3 times that of any dose amount or dose range listed above.

Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 60% (w/w) to about 70% (w/w), about 70% (w/w) to about 80% (w/w), or about 80% (w/w) to about 90% (w/w) of dextromethorphan.

(S)-Bupropion, (R)-bupropion, and/or dextromethorphan may be combined with a pharmaceutical carrier selected on the basis of the chosen route of administration and standard pharmaceutical practice as described, for example, in Remington's Pharmaceutical Sciences, 2005. The relative proportions of active ingredient and carrier may be determined, for example, by the solubility and chemical nature of the compounds, chosen route of administration, and standard pharmaceutical practice.

(S)-bupropion, (R)-bupropion, and/or dextromethorphan may be administered to a human patient in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal; ophthalmic; sublingual; and buccal, and topically including ophthalmic; dermal; ocular; rectal; and nasal.

(S)-bupropion, (R)-bupropion, and/or dextromethorphan may be formulated for oral administration, for example, with an inert diluent or with an edible carrier, or it may be enclosed in hard or soft shell gelatin capsules, compressed into tablets, or incorporated directly with the food of the diet. For oral therapeutic administration, the active compound may be incorporated with an excipient and used in the form of ingestible tablets, buccal tablets, troches, capsules, elixirs, suspensions, syrups, wafers, and the like.

Tablets, troches, pills, capsules and the like containing (S)-bupropion, (R)-bupropion, and/or dextromethorphan may also contain one or more of the following: a binder such as gum tragacanth, acacia, corn starch, or gelatin; an excipient, such as dicalcium phosphate; a disintegrating agent such as corn starch, potato starch, alginic acid, and the like; a lubricant such as magnesium stearate; a sweetening agent such as sucrose, lactose, or saccharin; or a flavoring agent such as peppermint, oil of wintergreen, or cherry flavoring. When the dosage form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. It may be desirable for material in a dosage form or pharmaceutical composition to be pharmaceutically pure and substantially non-toxic in the amounts employed.

Some compositions or dosage forms may be a liquid, or may comprise a solid phase dispersed in a liquid.

(S)-bupropion, (R)-bupropion, and/or dextromethorphan may be formulated for parental or intraperitoneal administration. Solutions of the active compounds as free bases or pharmacologically acceptable salts can be prepared in water suitably mixed with a surfactant. A dispersion can also have an oil dispersed within, or dispersed in, glycerol, liquid polyethylene glycols, and mixtures thereof. Under ordinary conditions of storage and use, these preparations may contain a preservative to prevent the growth of microorganisms.

A dosage form or a composition that contains both (S)-Bupropion, or (R)-bupropion, and dextromethorphan may be a blend or mixture of the bupropion and the dextromethorphan, either alone or within a vehicle. For example, (S)-Bupropion or (R)-bupropion and dextromethorphan may be dispersed within each other or dispersed together within a vehicle. A dispersion may include a mixture of solid materials wherein small individual particles are substantially one compound, but the small particles are dispersed within one another, such as might occur if two powders of two different drugs are blended with a solid vehicle material, and the blending is done in the solid form. In some embodiments, dextromethorphan and (S)-bupropion or (R)-bupropion may be substantially uniformly dispersed within a composition or dosage form. Alternatively, dextromethorphan and (S)-Bupropion or (R)-bupropion may be in separate domains or phases within a composition or dosage form. For example, one drug may be in a coating and another drug may be in a core within the coating. For example, one drug may be formulated for sustained release and another drug may be formulated for immediate release.

Some embodiments include administration of a tablet that contains (S)-Bupropion or (R)-bupropion in a form that provides sustained release, and dextromethorphan, if present, in a form that provides immediate release. While there are many ways that sustained release of bupropion may be achieved, in some embodiments, (S)-Bupropion or (R)-bupropion is combined with hydroxypropyl methylcellulose. For example, particles of (S)-Bupropion or (R)-bupropion could be blended with microcrystalline cellulose and hydroxypropyl methylcellulose (e.g., METHOCEL®) to form an admixture of blended powders. This could then be combined with immediate release dextromethorphan in a single tablet.

Administering a combination of (R)-bupropion and dextromethorphan to a human being in need of treatment by dextromethorphan may result in increased dextromethorphan levels, as compared to administering a combination of the same amount of (S)-bupropion and the same amount dextromethorphan in the same amounts. In some embodiments, the Cₘₐₓ, such as the average Cₘₐₓ, the mean Cₘₐₓ, the median Cₘₐₓ, or the Cₘₐₓ of an individual, or AUC₀₋₁₂, such as the average AUC₀₋₁₂, the mean AUC₀₋₁₂, the median AUC₀₋₁₂, or the AUC₀₋₁₂ of an individual, or AUC₀₋₁₂, such as the average AUC₀₋₁₂, the mean AUC₀₋₁₂, the median AUC₀₋₁₂, or the AUC₀₋₁₂ of dextromethorphan is increased by at least about 10%, at least about 20%, at least about 25%, at least about 30%, at least about 35%, at least about 40%, at least about 50%, at least about 80%, at least about 100%, at least about 150%, or at least about 200%, as compared to administering the same amount of (S)-bupropion and the same amount of dextromethorphan. This increase may be observed on day 1, 2, 3, 4, 5, 6, 7, or 8 of the treatment, or later. In some embodiments, the increase is observed on day 1. In some embodiments, the increase is observed on day 8.

In some embodiments, the combination of (R)-bupropion and dextromethorphan may achieve an AUC₀₋₁₂, such as the average AUC₀₋₁₂, the mean AUC₀₋₁₂, the median AUC₀₋₁₂, or the AUC₀₋₁₂ of an individual, of dextromethorphan, such as on days 1-60, that is about 30-150 ng·hr/mL, about 50-100 ng·hr/mL, about 80-100 ng·hr/mL, about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, about 1000 ng·hr/mL to about 1300 ng·hr/mL, or any AUC₀₋₁₂ of dextromethorphan in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 5 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 6 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 7 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 8 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, about 1000 ng·hr/mL to about 1300 ng·hr/mL, or any AUC₀₋₁₂ of dextromethorphan in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 9 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 10 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 11 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (R)-bupropion and dextromethorphan may achieve a Cₘₐₓ, such as the average Cₘₐₓ, the mean Cₘₐₓ, the median Cₘₐₓ, or the Cₘₐₓ of an individual, of dextromethorphan, such as on days 1-60, that is at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, about 2-20 ng/mL, 5-10 ng/mL, about 10-15 ng/mL, about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, about 100-110 ng/mL, or any Cₘₐₓ in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve a Cₘₐₓ, such as the average Cₘₐₓ, the mean Cₘₐₓ, the median Cₘₐₓ, or the Cₘₐₓ of an individual, of dextromethorphan on day 5 that is at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve a Cₘₐₓ, such as the average Cₘₐₓ, the mean Cₘₐₓ, the median Cₘₐₓ, or the Cₘₐₓ of an individual, of dextromethorphan on day 6 that is at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve a Cₘₐₓ, such as the average Cₘₐₓ, the mean Cₘₐₓ, the median Cₘₐₓ, or the Cₘₐₓ of an individual, of dextromethorphan on day 7 that is at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve a Cₘₐₓ, such as the average Cₘₐₓ, the mean Cₘₐₓ, the median Cₘₐₓ, or the Cₘₐₓ of an individual, of dextromethorphan on day 8 that is at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, about 100-110 ng/mL, or any Cₘₐₓ in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve a Cₘₐₓ, such as the average Cₘₐₓ, the mean Cₘₐₓ, the median Cₘₐₓ, or the Cₘₐₓ of an individual, of dextromethorphan on day 9 that is at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve a Cₘₐₓ, such as the average Cₘₐₓ, the mean Cₘₐₓ, the median Cₘₐₓ, or the Cₘₐₓ of an individual, of dextromethorphan on day 10 that is at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve a Cₘₐₓ, such as the average Cₘₐₓ, the mean Cₘₐₓ, the median Cₘₐₓ, or the Cₘₐₓ of an individual, of dextromethorphan on day 11 that is at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

The dextromethorphan fluctuation index values FI(%) can be determined by equation:

$$FI ( \% ) = \frac{( {C}_{max} - {C}_{min} )}{{C}_{avg}} \times 100.$$

In some embodiments, the combination of (R)-bupropion and dextromethorphan, for example by twice daily administration of about 30-60 mg of dextromethorphan, and twice daily administration of about 50-150 mg of (R)-bupropion, may achieve an FI(%) value, such as the average FI(%) value, the mean FI(%) value, the median FI(%) value, or the FI(%) value of an individual, of dextromethorphan on day 8 that is less than 100%, less than 50%, less than 40%, less than 30%, about 20-50%, about 20-40%, about 20-30%, or any FI(%) value in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve an FI(%) value, such as the average FI(%) value, the mean FI(%) value, the median FI(%) value, or the FI(%) value of an individual, of dextromethorphan on day 9 that is less than 100%, less than 50%, less than 40%, less than 30%, about 20-50%, about 20-40%, about 20-30%, or any FI(%) value in a range bounded by any of these values.

In some embodiments, the combination of (R)-bupropion and dextromethorphan, for example by twice daily administration of about 30-60 mg of dextromethorphan, and twice daily administration of about 50-150 mg of (R)-bupropion, may achieve an FI(%) value, such as the average FI(%) value, the mean FI(%) value, the median FI(%) value, or the FI(%) value of an individual, of dextromethorphan on day 10 that is less than 100%, less than 50%, less than 40%, less than 30%, about 20-50%, about 20-40%, about 20-30%, or any FI(%) value in a range bounded by any of these values.

Although administering a combination of (R)-bupropion and dextromethorphan to a human being in need of treatment by dextromethorphan may result in increased dextromethorphan levels, as compared to administering a combination of (S)-bupropion and dextromethorphan in the same amounts, administering a combination of (S)-bupropion and dextromethorphan can still result in an increase of dextromethorphan plasma levels as compared to administering dextromethorphan without administering a (S)-bupropion.

In some embodiments, administering a combination of (R)-bupropion and dextromethorphan to a human being in need of treatment by dextromethorphan may result in increased dextromethorphan levels, as compared to administering a combination of racemic-bupropion and dextromethorphan in the same amounts. In some embodiments, such an increase can be significant or substantial.

In some embodiments, the combination of (S)-bupropion and dextromethorphan may achieve an AUC₀₋₁₂, such as the average AUC₀₋₁₂, the mean AUC₀₋₁₂, the median AUC₀₋₁₂, or the AUC₀₋₁₂ of an individual, of dextromethorphan, such as on days 1-60, that is about 10-50 ng·hr/mL, about 20-40 ng·hr/mL, about 30-100 ng·hr/mL, about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, about 1000-1100 ng·hr/mL, or any AUC₀₋₁₂ of dextromethorphan in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 5 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 6 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 7 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 8 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, about 1000-1100 ng·hr/mL, or any AUC₀₋₁₂ of dextromethorphan in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 9 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 10 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve an AUC₀₋₁₂, such as the average AUC₀₋₁₂, the mean AUC₀₋₁₂, the median AUC₀₋₁₂, or the AUC₀₋₁₂ of an individual, of dextromethorphan on day 11 that is about 400-425 ng·hr/mL, about 425-450 ng·hr/mL, about 400-450 ng·hr/mL, about 450-475 ng·hr/mL, about 475-500 ng·hr/mL, about 450-500 ng·hr/mL, about 500-510 ng·hr/mL, about 510-520 ng·hr/mL, about 520-530 ng·hr/mL, about 530-540 ng·hr/mL, about 540-560 ng·hr/mL, about 500-550 ng·hr/mL, about 550-575 ng·hr/mL, about 575-600 ng·hr/mL, about 550-600 ng·hr/mL, about 600-650 ng·hr/mL, about 650-700 ng·hr/mL, about 700-750 ng·hr/mL, about 750-800 ng·hr/mL, about 800-820 ng·hr/mL, about 820-840 ng·hr/mL, about 840-850 ng·hr/mL, about 800-850 ng·hr/mL, about 850-860 ng·hr/mL, about 860-870 ng·hr/mL, about 870-880 ng·hr/mL, about 880-890 ng·hr/mL, about 890-900 ng·hr/mL, about 850-900 ng·hr/mL, about 900-910 ng·hr/mL, about 910-930 ng·hr/mL, about 930-950 ng·hr/mL, about 900-950 ng·hr/mL, about 950-1000 ng·hr/mL, or any AUC₀₋₁₂ of dextromethorphan in a range bounded by any of these values.

In some embodiments, the combination of (S)-bupropion and dextromethorphan may achieve a Cₘₐₓ, such as the average Cₘₐₓ, the mean Cₘₐₓ, the median Cₘₐₓ, or the Cₘₐₓ of an individual, of dextromethorphan, such as on days 1-60, that is about 1-10 ng/mL, about 2-5 ng/mL, about 5-10 ng/mL, about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, about 100-110 ng/mL, or any Cₘₐₓ in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve a Cₘₐₓ, such as the average Cₘₐₓ, the mean Cₘₐₓ, the median Cₘₐₓ, or the Cₘₐₓ of an individual, of dextromethorphan on day 5 that is about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve a Cₘₐₓ, such as the average Cₘₐₓ, the mean Cₘₐₓ, the median Cₘₐₓ, or the Cₘₐₓ of an individual, of dextromethorphan on day 6 that is about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve a Cₘₐₓ, such as the average Cₘₐₓ, the mean Cₘₐₓ, the median Cₘₐₓ, or the Cₘₐₓ of an individual, of dextromethorphan on day 7 that is about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve a Cₘₐₓ, such as the average Cₘₐₓ, the mean Cₘₐₓ, the median Cₘₐₓ, or the Cₘₐₓ of an individual, of dextromethorphan on day 8 that is about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, about 100-110 ng/mL, or any Cₘₐₓ in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve a Cₘₐₓ, such as the average Cₘₐₓ, the mean Cₘₐₓ, the median Cₘₐₓ, or the Cₘₐₓ of an individual, of dextromethorphan on day 9 that is about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve a Cₘₐₓ, such as the average Cₘₐₓ, the mean Cₘₐₓ, the median Cₘₐₓ, or the Cₘₐₓ of an individual, of dextromethorphan on day 10 that is about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve a Cₘₐₓ, such as the average Cₘₐₓ, the mean Cₘₐₓ, the median Cₘₐₓ, or the Cₘₐₓ of an individual, of dextromethorphan on day 11 that is about 40-43 ng/mL, about 43-45 ng/mL, about 40-45 ng/mL, about 45-48 ng/mL, about 48-50 ng/mL, about 45-50 ng/mL, about 50-51 ng/mL, about 51-52 ng/mL, about 52-53 ng/mL, about 53-54 ng/mL, about 54-56 ng/mL, about 50-55 ng/mL, about 55-58 ng/mL, about 58-60 ng/mL, about 55-60 ng/mL, about 60-65 ng/mL, about 65-70 ng/mL, about 70-75 ng/mL, about 75-80 ng/mL, about 80-82 ng/mL, about 82-84 ng/mL, about 84-85 ng/mL, about 80-85 ng/mL, about 85-86 ng/mL, about 86-87 ng/mL, about 87-88 ng/mL, about 88-89 ng/mL, about 89-90 ng/mL, about 85-90 ng/mL, about 90-91 ng/mL, about 91-93 ng/mL, about 93-95 ng/mL, about 90-95 ng/mL, about 95-100 ng/mL, or any Cₘₐₓ in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve an FI(%) value, such as the average FI(%) value, the mean FI(%) value, the median FI(%) value, or the FI(%) value of an individual, of dextromethorphan on day 8 that is less than 100%, less than 50%, less than 40%, less than 30%, about 20-50%, about 20-40%, about 20-30%, or any FI(%) value in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve an FI(%) value, such as the average FI(%) value, the mean FI(%) value, the median FI(%) value, or the FI(%) value of an individual, of dextromethorphan on day 9 that is less than 100%, less than 50%, less than 40%, less than 30%, about 20-50%, about 20-40%, about 20-30%, or any FI(%) value in a range bounded by any of these values.

In some embodiments, the combination of (S)-bupropion and dextromethorphan, for example by twice daily administration of about 30-60 mg of dextromethorphan, and twice daily administration of about 50-150 mg of (S)-bupropion, may achieve an FI(%) value, such as the average FI(%) value, the mean FI(%) value, the median FI(%) value, or the FI(%) value of an individual, of dextromethorphan on day 10 that is less than 100%, less than 50%, less than 40%, less than 30%, about 20-50%, about 20-40%, about 20-30%, or any FI(%) value in a range bounded by any of these values.

Examples of neurological disorders or central nervous system disorders that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to: affective disorders, psychiatric disorders, cerebral function disorders, movement disorders, dementias, motor neuron diseases, neurodegenerative diseases, seizure disorders, and headaches.

Affective disorders that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to, depression, major depression, treatment resistant depression and treatment resistant bipolar depression, bipolar disorders including cyclothymia, seasonal affective disorder, mood disorders, chronic depression (dysthymia), psychotic depression, postpartum depression, premenstrual dysphoric disorder (PMDD), situational depression, atypical depression, mania, anxiety disorders, attention deficit disorder (ADD), attention deficit disorder with hyperactivity (ADDH), and attention deficit/hyperactivity disorder (AD/HD), bipolar and manic conditions, obsessive-compulsive disorder, bulimia, obesity or weight-gain, narcolepsy, chronic fatigue syndrome, premenstrual syndrome, an addictive disorder, substance addiction or abuse, nicotine addiction, psycho-sexual dysfunction, pseudobulbar affect, and emotional lability.

Depression may be manifested by depressive symptoms. These symptoms may include psychological changes such as changes in mood, feelings of intense sadness, despair, mental slowing, loss of concentration, pessimistic worry, agitation, anxiety, irritability, guilt, anger, feelings of worthlessness, reckless behavior, suicidal thoughts or attempts, and/or self-deprecation. Physical symptoms of depression may include insomnia, anorexia, appetite loss, weight loss, weight gain, decreased energy and libido, fatigue, restlessness, aches, pains, headaches, cramps, digestive issues, and/or abnormal hormonal circadian rhythms.

Some patients, even after treatment with medications such as antidepressants, may have an inadequate or no response to the treatment. Treatment resistant depression (TRD), or treatment-refractory depression, is a condition generally associated with patients who have failed treatment with at least two antidepressants. Part of the diagnosis for TRD is for the patient to have had an inadequate response to treatment with the antidepressants after an adequate dose and adequate course. TRD may be more difficult to treat due to the comorbidity of other medical or psychological illnesses, such as drug/alcohol abuse or eating disorders, or TRD being misdiagnosed. Some TRD patients have had an inadequate response to 1, 2, 3, or more adequate antidepressant treatment trials or have failed or had an inadequate response to 1, 2, 3, or more prior antidepressant treatments. In some embodiments, a patient being treated for treatment resistant depression has failed treatment with at least 1, 2, 3, 4, 5, 6, 7, 8, 9, 10, or more antidepressant therapies.

Measures of treatment effect that may be improved by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan, include, but are not limited to: Montgomery-Asberg Depression Rating Scale (MADRS), Quality of Life Enjoyment and Satisfaction Questionnaire Short Form, Range of Impaired Functioning Tool, Sheehan Disability Scale, Patient Rated Inventory of Side Effects (PRISE), Columbia-Suicide Severity Rating Scale (C-SSRS), Quick Inventory of Depressive Symptomatology, Self-Report (QID(S)-SR), Clinical Global Impression (CGI) scale, Massachusetts General Hospital Cognitive and Physical Functioning Questionnaire (CPFQ), 17-item Hamilton Rating Scale for Depression (HAM-D17), Massachusetts General Hospital Antidepressant Treatment Response Questionnaire (MGH ATRQ), 16-item Quick Inventory of Depressive Symptomatology—Self Report (QID(S)-5R16), Sheehan Disability Scale (SDS), Clinical Global Impression of Severity of Illness (CGI-S), Clinical Global Impression of Change (CGI-C), EuroQOL 5 Dimension 5 Level (EQ-5D-5L), Patient Global Impression of Change (PGIC), 7-item Generalized Anxiety Disorder (GAD-7), Clinical Global Impressions Improvement (CGI-I). Sheehan Disability Scale (SDS). 16-item Quick Inventory of Depressive Symptomatology—Self Report (QID(S)-5R16), Hamilton Anxiety Scale (HAM-A), Massachusetts General Hospital Cognitive and Physical Functioning Questionnaire (CPFQ), CPFQ—Cognitive subscales (Items 4 to 7), Brief Psychiatric Rating Scale (BPRS), etc.; Digit Symbol Substitution Test (DSST), Rey Auditory Verbal Learning Task (RAVLT), Trail Making Test (TMT), Stroop Colour Naming Test (STROOP), Simple Reaction Time (SRT), Choice Reaction Time (CRT), etc.

Patients who may benefit from the treatments described herein include pediatric patients, such as patients under about 18 years of age, about 0-5 years of age, about 5-10 years of age, about 10-12 years of age, or about 12-18 years of age, adult patients, such as patients having an age of about 18-65 years; about 18-30 years; about 30-50 years; about 50-65 years, and elderly patients, such as patients over 65 years of age; about 65-75 years of age; about 75-90 years of age; or over 90 years of age.

Treatment of TRD using enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan may result in a reduction of depressive symptoms of at least about 5%, at least about 10%, at least about 20%, at least about 30%, at least about 40%, at least about 50%, at least about 60%, at least about 70%, at least about 80%, at least about 90%, up to about 100%, or any other reduction percentage in a range bounded by any of these values.

Psychiatric disorders that may be treated using enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to, anxiety disorders, such as phobias, generalized anxiety disorder, social anxiety disorder, panic disorder, agoraphobia, obsessive-compulsive disorder, and post-traumatic stress disorder (PTSD); mania, manic depressive illness, hypomania, unipolar depression, depression, stress disorders, somatoform disorders, personality disorders, psychosis, schizophrenia, delusional disorder, schizoaffective disorder, schizotypy, aggression, aggression in Alzheimer's disease, agitation, and agitation in Alzheimer's disease. Agitation associated with Alzheimer's disease occurs as the disease progresses. Agitation may present itself as inappropriate verbal, emotional, and/or physical behaviors. Inappropriate behaviors may include, but are not limited to, incoherent babbling, inappropriate emotional response, demands for attention, threats, irritability, frustration, screaming, repetitive questions, mood swings, cursing, abusive language, physical outbursts, emotional distress, restlessness, shredding, sleeping disturbances, delusions, hallucinations, pacing, wandering, searching, rummaging, repetitive body motions, hoarding, shadowing, hitting, scratching, biting, combativeness, hyperactivity, and/or kicking.

In some embodiments, treatment of agitation associated with Alzheimer's disease using enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan may result in a reduction of agitation-related symptoms of at least about 5%, at least about 10%, at least about 20%, at least about 30%, at least about 40%, at least about 50%, at least about 60%, at least about 70%, at least about 80%, at least about 90%, up to about 100%, or any other reduction percentage in a range bounded by any of these values.

Measures of treatment effect of agitation that may be improved by treatment enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to, Neuropsychiatric Inventory-Clinician (NPI-C) rating scale, overall and all domains; Neuropsychiatric Inventory-Clinician (NPI-C) rating scale Agitation domain; Cohen-Mansfield Agitation Inventory (CMAI); Cornell Scale for Depression in Dementia (CSDD); Neuropsychiatric Inventory (NPI Agitation/Aggression Domain); Cocomitant Medications (Frequency of using concomitant medications); Alzheimer's Disease Cooperative Study—Activities of Daily Living Inventory (ADC(S)-ADL); Neuropsychiatric Inventory (NPI) Individual Domains and NPI Total Scores (range 0-144), including NPI-C Apathy domain, NPI Agitation/Aggression Caregiver Distress, Modified Alzheimer's Disease Cooperative Study-Clinical Global Impression of Change Agitation (mADC(S)-CGIC Agitation), Patient Global Impression of Change (PGIC) (rated by caregiver), Dementia Quality of Life (DEMQOL), Quality of Life-Alzheimer's disease measure (QoL-AD), Zarit Burden Scale, Resource Utilization in Dementia (RUD), Alzheimer's Disease Assessment Scale-Cognitive Subscale (ADA(S)-Cog), Mini-mental State Examination (MMSE), Caregiver Strain Index (CSI), Individual Domain of the Neuropsychiatric Inventory (NPI), Total Neuropsychiatric Inventory (NPI) Score, Neuropsychiatric Inventory (Agitation/Aggression Domain of NPI), Neuropsychiatric Inventory (Caregiver Distress for NPI Domains), etc.

Substance addiction abuse that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan includes, but is not limited to, drug dependence, addiction to cocaine, psychostimulants (e.g., crack, cocaine, speed, meth), nicotine, alcohol, opioids, anxiolytic and hypnotic drugs, cannabis (marijuana), amphetamines, hallucinogens, phencyclidine, volatile solvents, and volatile nitrites. Nicotine addiction includes nicotine addiction of all known forms, such as smoking cigarettes, cigars and/or pipes, electronic cigarettes, and addiction to chewing tobacco.

Cerebral function disorders that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to, disorders involving intellectual deficits such as senile dementia, Alzheimer's type dementia, memory loss, amnesia/amnestic syndrome, epilepsy, disturbances of consciousness, coma, lowering of attention, speech disorders, voice spasms, Parkinson's disease, Lennox-Gastaut syndrome, autism, hyperkinetic syndrome, and schizophrenia. Cerebral function disorders also include disorders caused by cerebrovascular diseases including, but not limited to, stroke, cerebral infarction, cerebral bleeding, cerebral arteriosclerosis, cerebral venous thrombosis, head injuries, and the like where symptoms include disturbance of consciousness, senile dementia, coma, lowering of attention, and speech disorders.

Movement disorders that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to, akathisia, akinesia, associated movements, athetosis, ataxia, ballismus, hemiballismus, bradykinesia, cerebral palsy, chorea, Huntington's disease, rheumatic chorea, Sydenham's chorea, dyskinesia, tardive dyskinesia, dystonia, blepharospasm, spasmodic torticollis, dopamine-responsive dystonia, Parkinson's disease, restless legs syndrome (RLS), tremor, essential tremor, and Tourette's syndrome, and Wilson's disease.

Dementias that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to, Alzheimer's disease, Parkinson's disease, vascular dementia, dementia with Lewy bodies, mixed dementia, fronto-temporal dementia, Creutzfeldt-Jakob disease, normal pressure hydrocephalus, Huntington's disease, Wernicke-Korsakoff Syndrome, and Pick's disease. Motor neuron diseases that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to, amyotrophic lateral sclerosis (ALS), progressive bulbar palsy, primary lateral sclerosis (PLS), progressive muscular atrophy, post-polio syndrome (PPS), spinal muscular atrophy (SMA), spinal motor atrophies, Tay-Sach's disease, Sandoff disease, and hereditary spastic paraplegia.

Neurodegenerative diseases that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to Alzheimer's disease, prion-related diseases, cerebellar ataxia, spinocerebellar ataxia (SCA), spinal muscular atrophy (SMA), bulbar muscular atrophy, Friedrich's ataxia, Huntington's disease, Lewy body disease, Parkinson's disease, amyotrophic lateral sclerosis (ALS or Lou Gehrig's disease), multiple sclerosis (MS), multiple system atrophy, Shy-Drager syndrome, corticobasal degeneration, progressive supranuclear palsy, Wilson's disease, Menkes disease, adrenoleukodystrophy, cerebral autosomal dominant arteriopathy with subcortical infarcts and leukoencephalopathy (CADASIL), muscular dystrophies, Charcot-Marie-Tooth disease (CMT), familial spastic paraparesis, neurofibromatosis, olivopontine cerebellar atrophy or degeneration, striatonigral degeneration, Guillain-Barr syndrome, and spastic paraplegia.

Seizure disorders that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to, epileptic seizures, nonepileptic seizures, epilepsy, febrile seizures; partial seizures including, but not limited to, simple partial seizures, Jacksonian seizures, complex partial seizures, and epilepsia partialis continua; generalized seizures including, but not limited to, generalized tonic-clonic seizures, absence seizures, atonic seizures, myoclonic seizures, juvenile myoclonic seizures, and infantile spasms; and status epilepticus.

Types of headaches that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, but are not limited to, migraine, tension, and cluster headaches.

Other neurological disorders that may be treated by enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan include, Rett Syndrome, autism, tinnitus, disturbances of consciousness disorders, sexual dysfunction, intractable coughing, narcolepsy, cataplexy; voice disorders due to uncontrolled laryngeal muscle spasms, including, but not limited to, abductor spasmodic dysphonia, adductor spasmodic dysphonia, muscular tension dysphonia, and vocal tremor; diabetic neuropathy, chemotherapy-induced neurotoxicity, such as methotrexate neurotoxicity; incontinence including, but not limited, stress urinary incontinence, urge urinary incontinence, and fecal incontinence; and erectile dysfunction.

In some embodiments, enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan may be used to treat pain, joint pain, pain associated with sickle cell disease, pseudobulbar affect, depression (including treatment resistant depression), disorders related to memory and cognition, schizophrenia, Parkinson's disease, amyotrophic lateral sclerosis (ALS), Rhett's syndrome, seizures, cough (including chronic cough), etc.

In some embodiments, enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan may be used to treat treatment refractory depression.

Enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan may be used to treat, or provide relief to, any type of pain including, but not limited to, musculoskeletal pain, neuropathic pain, cancer-related pain, acute pain, nociceptive pain, inflammatory pain, arthritis pain, complex regional pain syndrome, etc.

In some embodiments, enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan may be useful to relieve neuropathic pain.

Examples of neuropathic pain include diabetic peripheral neuropathy, post-herpetic neuralgia, trigeminal neuralgia, monoradiculopathies, phantom limb pain, central pain, etc. Other causes of neuropathic pain include cancer-related pain, lumbar nerve root compression, spinal cord injury, post-stroke pain, central multiple sclerosis pain, HIV-associated neuropathy, and radio- or chemo-therapy associated neuropathy, etc.

In some embodiments, enhanced bioavailability or enhanced plasma levels of (S)-bupropion, enhanced bioavailability or enhanced plasma levels of (R,R)-hydroxybupropion, and/or enhanced bioavailability or enhanced plasma levels of dextromethorphan may be administered to relieve fibromyalgia.

Adverse events associated with bupropion or dextromethorphan that may be avoided or reduced by a method described herein include a central nervous system adverse event, a gastrointestinal event, or another type of adverse event associated with any of these compounds. Central nervous system (CNS) adverse events include, but are not limited to, nervousness, dizziness, sleeplessness, light-headedness, tremor, hallucinations, convulsions, CNS depression, fear, anxiety, headache, increased irritability or excitement, tinnitus, drowsiness, dizziness, sedation, somnolence, confusion, disorientation, lassitude, incoordination, fatigue, euphoria, nervousness, insomnia, sleeping disturbances, convulsive seizures, excitation, catatonic-like states, hysteria, hallucinations, delusions, paranoia, headaches and/or migraine, and extrapyramidal symptoms such as oculogyric crisis, torticollis, hyperexcitability, increased muscle tone, ataxia, and/or tongue protrusion.

Gastrointestinal adverse events include, but are not limited to, nausea, vomiting, abdominal pain, dysphagia, dyspepsia, diarrhea, abdominal distension, flatulence, peptic ulcers with bleeding, loose stools, constipation, stomach pain, heartburn, gas, loss of appetite, feeling of fullness in stomach, indigestion, bloating, hyperacidity, dry mouth, gastrointestinal disturbances, and gastric pain.

Other adverse events that may be reduced or avoided by a method described herein include abnormal sensation of rotation and movement, agitation, arm weakness, bloating, blurred vision, burning sensation in the eyes, buzzing sound in ear, changes in vital signs (including, but not limited to, heart rate, respiratory rate, body temperature, blood pressure), cold sensation, constipation, difficulty concentrating, difficulty sleeping, difficulty in falling asleep, difficulty urinating, difficulty with bowel movement, discomfort in the ear, discomfort in the eye, discomfort in the stomach, dizziness, dry lips, dry mouth, dry throat, dysmenorrhea, fatigue, feeling feverish, feeling heavy headed, feeling more agitated than usual, feeling more tired than usual, feeling tired, hand tremors, hand weakness, headache, heartburn, hot flashes, increased blood pressure, increased skin sensitivity, increased skin sensitivity at head and face, involuntary muscle contraction, involuntary muscle contractions at all over the body, knee pain, leg weakness, lightheadedness, loose stool, loss of appetite, low back pain, menstrual disorder, metallic taste, more saliva than usual, mucosal dryness, nasal congestion, nausea, runny nose, sensation of light pressure sensation in the eyes, shivers when stretching or yawning, skin sensitivity, skin sensitivity in arm, face, and/or head, sleep difficulties, soft stools, stomach ache, stomach discomfort, sweaty hands and/or feet, throat irritation, throat pain, tinnitus, tremors, and/or weakness. Any of these side effects may also be referred to, or grouped, according to a corresponding, equivalent, or otherwise relevant term found in the Medical Dictionary for Regulatory Activities (MedRA).

The term “treating” or “treatment” includes the diagnosis, cure, mitigation, treatment, or prevention of disease in man or other animals, or any activity that otherwise affects the structure or any function of the body of man or other animals.

Patients who may benefit from the treatments described herein include pediatric patients, such as patients under about 18 years of age, about 0-5 years of age, about 5-10 years of age, about 10-12 years of age, or about 12-18 years of age; adult patients, such as patients having an age of about 18-65 years, about 18-30 years, about 30-50 years, about 50-65 years; and elderly patients, such as patients over 65 years of age, about 65-75 years of age, about 75-90 years of age, or over 90 years of age.

U.S. Pat. No. 9,867,819, issued on Jan. 16, 2018 to Herriot Tabuteau under the title “Compositions and methods for increasing the metabolic lifetime of dextromethorphan and related pharmacodynamics effects” from application Ser. No. 15/645,939, which was filed on Jul. 10, 2017, is incorporated by reference herein in its entirety. In particular, the amounts and dosing regimens described for bupropion in U.S. Pat. No. 9,867,819 can be applied to (S)-bupropion or (R)-bupropion described herein. Additionally, the amounts and dosing regimens for dextromethorphan described in U.S. Pat. No. 9,867,819 can be applied herein. Furthermore, the conditions that may be treated with the dosage forms described in the U.S. Pat. No. 9,867,819 may also be treated in a similar fashion using the dosage forms and methods described herein.

The following embodiments are contemplated:

### Embodiment 1

A method of delivering a bupropion or a metabolite thereof to plasma comprising orally administering a dosage form containing about 50 mg to about 100 mg of (S)-bupropion that is at least 95% enantiomerically pure, at least once per day, to a human being.

### Embodiment 2

A method of providing a bupropion to the plasma of a human being, comprising:
selecting a human patient in need of a pharmacokinetic profile provided by orally administering a reference dosage form containing a first amount of racemic bupropion at a first dosing frequency; and
orally administering a dosage form containing a second amount of (S)-bupropion that is at least 95% enantiomerically pure at the first dosing frequency to achieve the same pharmacokinetic profile that would be achieved by administering the reference dosage form at the first dosing frequency;
wherein the first dosing frequency is once daily or twice daily; and wherein the second amount is about 40% to about 60% of the first amount.

### Embodiment 3

A method of treating a condition that is treatable with racemic bupropion, comprising:
selecting a human patient having the condition that is treatable by orally administering a reference dosage form containing a first amount of racemic bupropion at a first dosing frequency; and
orally administering a dosage form containing a second amount of (S)-bupropion that is at least 95% enantiomerically pure at the first dosing frequency to achieve the same therapeutic effect that would be achieved by administering the reference dosage form at the first dosing frequency;
wherein the first dosing frequency is once daily or twice daily; and
wherein the second amount is about 40% to about 60% of the first amount.

### Embodiment 4

The method of embodiment 1, wherein the human being is in need of treatment with (S)-bupropion.

### Embodiment 5

The method of embodiment 1, 2, 3, or 4, wherein the method achieves a Cₘₐₓ of (S)-bupropion that is at least about 60 ng/mL.

### Embodiment 6

The method of embodiment 1, 2, 3, 4, or 5, wherein the method is effective in increasing the Cₘₐₓ of (S)-bupropion at least 5-fold as compared to the Cₘₐₓ of (R)-bupropion that results from administering the same amount of (R)-bupropion to the human being.

### Embodiment 7

The method of embodiment 1, 2, 3, 4, 5, or 6, wherein the method achieves a Cₘₐₓ of (R,R)-hydroxybupropion that is at least about 500 ng/mL in the human being.

### Embodiment 8

The method of embodiment 1, 2, 3, 4, 5, 6, or 7, wherein the method is effective in increasing the Cₘᵢₙ of (R,R)-hydroxybupropion at least 3-fold as compared to the Cₘᵢₙ of (R,R)-hydroxybupropion that results from administering the same amount of (R)-bupropion to the human being.

### Embodiment 9

The method of embodiment 1, 2, 3, 4, 5, 6, 7, or 8, wherein the dosage form is administered once daily.

### Embodiment 10

The method of embodiment 1, 2, 3, 4, 5, 6, 7, or 8, wherein the dosage form is administered twice daily.

### Embodiment 11

The method of embodiment 1, 2, 3, 4, 5, 6, 7, 8, 9, or 10, wherein (R,R)-hydroxybupropion is at least 97% of the total of amount of (R,R)-hydroxybupropion and (S,S)-hydroxybupropion present in the plasma of the human being.

### Embodiment 12

The method of embodiment 1, 2, 3, 4, 5, 6, 7, 8, 9, 10, or 11, which is effective in providing therapeutically effective plasma levels of (R,R)-hydroxybupropion.

### Embodiment 13

The method of embodiment 1, 2, 3, 4, 5, 6, 7, 8, 9, 10, 11, or 12, which is effective in providing therapeutically effective plasma levels of (S)-bupropion.

### Embodiment 14

The method of embodiment 1, 2, 3, 4, 5, 6, 7, 8, 9, 10, 11, 12, or 13, wherein the human being is in need of treatment with (R,R)-hydroxybupropion.

### Embodiment 15

The method of embodiment 1, 2, 3, 4, 5, 6, 7, 8, 9, 10, 11, 12, 13, or 14, wherein the method achieves a Cₘₐₓ of (R,R)-hydroxybupropion that is at least about 500 ng/mL in the human being.

### Embodiment 16

The method of embodiment 1, 2, 3, 4, 5, 6, 7, 8, 9, 10, 11, 12, 13, 14, or 15, wherein the dosage form is administered for at least 8 consecutive days.

### Embodiment 17

The method of embodiment 16, wherein the dosage form is administered for at least 14 consecutive days.

### Embodiment 18

The method of embodiment 16, wherein the dosage form is administered for at least 21 consecutive days.

### Embodiment 19

The method of embodiment 1, 2, 3, 4, 5, 6, 7, 8, 9, 10, 11, 12, 13, 14, 15, 16, 17, or 18, wherein the dosage form contains about 60 mg to about 90 mg of (S)-bupropion.

### Embodiment 20

The method of embodiment 1, 2, 3, 4, 5, 6, 7, 8, 9, 10, 11, 12, 13, 14, 15, 16, 17, 18, or 19, wherein the dosage form is administered once daily for 1 to 3 consecutive days, then the dosage form is administered twice a day for at least the following 4 to 7 consecutive days, so that the dosage form is administered once daily or twice daily for a total of at least 8 consecutive days.

### Embodiment 21

The method of embodiment 1, 2, 3, 4, 5, 6, 7, 8, 9, 10, 11, 12, 13, 14, 15, 16, 17, 18, 19, or 20, wherein the dosage form is administered once daily for 1 to 7 consecutive days, then the dosage form is administered twice a day for at least the following 7 consecutive days, so that the dosage form is administered once daily or twice daily for a total of at least 8 consecutive days.

### Embodiment 22

The method of embodiment 1, 2, 3, 4, 5, 6, 7, 8, 9, 10, 11, 12, 13, 14, 15, 16, 17, 18, 19, 20, or 21, wherein the dosage form contains about 70 mg to about 80 mg of the (S)-bupropion.

### Embodiment 23

The method of embodiment 1, 2, 3, 4, 5, 6, 7, 8, 9, 10, 11, 12, 13, 14, 15, 16, 17, 18, 19, 20, 21, or 22, wherein the method achieves a Cₘₐₓ of (S)-bupropion in the human being that is at least about 70 ng/mL.

### Embodiment 24

The method of embodiment 1, 2, 3, 4, 5, 6, 7, 8, 9, 10, 11, 12, 13, 14, 15, 16, 17, 18, 19, 20, 21, 22, or 23, wherein the method achieves an AUC₀₋₁₂ of (S)-bupropion in the human being that is at least about 400 ng·hr/mL.

### Embodiment 25

The method of embodiment 24, wherein the method achieves an AUC₀₋₁₂ of (S)-bupropion in the human being that is about 500 ng·hr/mL to about 900 ng·hr/mL.

### Embodiment 26

The method of embodiment 1, 2, 3, 4, 5, 6, 7, 8, 9, 10, 11, 12, 13, 14, 15, 16, 17, 18, 19, 20, 21, 22, 23, 24, or 25, wherein the dosage form provides sustained release of the (S)-bupropion.

### Embodiment 27

The method of embodiment 1, 2, 3, 4, 5, 6, 7, 8, 9, 10, 11, 12, 13, 14, 15, 16, 17, 18, 19, 20, 21, 22, 23, 24, 25, or 26, wherein the dosage form further contains dextromethorphan.

### Embodiment 28

The method of embodiment 1, 2, 3, 4, 5, 6, 7, 8, 9, 10, 11, 12, 13, 14, 15, 16, 17, 18, 19, 20, 21, 22, 23, 24, 25, 26, or 27, wherein the dosage form contains about 70 mg to about 80 mg of the (S)-bupropion.

### Embodiment 29

The method of embodiment 1, 2, 3, 4, 5, 6, 7, 8, 9, 10, 11, 12, 13, 14, 15, 16, 17, 18, 19, 20, 21, 22, 23, 24, 25, 26, 27, or 28, wherein the method achieves a Cₘₐₓ of (R,R)-hydroxybupropion in the human being that is at least about 600 ng/mL.

### Embodiment 30

The method of embodiment 1, 2, 3, 4, 5, 6, 7, 8, 9, 10, 11, 12, 13, 14, 15, 16, 17, 18, 19, 20, 21, 22, 23, 24, 25, 26, 27, 28, or 29, wherein the method achieves an AUC₁₋₁₂ of (R,R)-hydroxybupropion in the human being that is at least about 7000 ng·hr/mL.

### Embodiment 31

The method of embodiment 30, wherein the method achieves an AUC₀₋₁₂ of (R,R)-hydroxybupropion in the human being that is at least about 8000 ng·hr/mL.

### Embodiment 32

A method of enhancing the plasma levels of (S)-bupropion and dextromethorphan, comprising orally co-administering, at least once per day, dextromethorphan and at least about 90 mg of (S)-bupropion that is at least 95% enantiomerically pure, to a human being in need of treatment with both (S)-bupropion and dextromethorphan, wherein the method achieves a Cₘₐₓ of (S)-bupropion that is at least about 90 ng/mL in the human being, wherein the method is effective in increasing the Cₘₐₓ of (S)-bupropion at least 3-fold as compared the Cₘₐₓ of (R)-bupropion that results from administering the same of amount of (R)-bupropion to the human being.

### Embodiment 33

The method of embodiment 32, wherein the dextromethorphan and the (S)-bupropion are co-administered for at least 8 consecutive days.

### Embodiment 34

The method of embodiment 32, wherein the dextromethorphan and the (S)-bupropion are co-administered for at least 14 consecutive days.

### Embodiment 35

The method of embodiment 32, wherein the dextromethorphan and the (S)-bupropion are co-administered in a single dosage form.

### Embodiment 36

The method of embodiment 33, 34, or 35, wherein the dosage form is administered once daily for 1 to 3 consecutive days, then the dosage form is administered twice a day for at least the following 4 to 7 consecutive days, so that the dosage form is administered once daily or twice daily for a total of at least 8 consecutive days.

### Embodiment 37

The method of embodiment 33, 34, or 35, wherein the dosage form is administered once daily for 1 to 7 consecutive days, then the dosage form is administered twice a day for at least the following 7 consecutive days, so that the dosage form is administered once daily or twice daily for a total of at least 8 consecutive days.

### Embodiment 38

The method of embodiment 32, 33, 34, 35, 36, or 37, wherein (S)-bupropion is administered in a dosage form containing about 100 mg to about 110 mg of the (S)-bupropion.

### Embodiment 39

The method of embodiment 32, 33, 34, 35, 36, 37, or 38, wherein the Cₘₐₓ of (S)-bupropion in the human being that is at least about 110 ng/mL.

### Embodiment 40

The method of embodiment 32, 33, 34, 35, 36, 37, 38, or 39, wherein the method achieves an AUC₀₋₁₂of (S)-bupropion in the human being is at least about 800 ng·hr/mL.

### Embodiment 41

The method of embodiment 32, 33, 34, 35, 36, 37, 38, 39, or 40, wherein (S)-bupropion is administered in a dosage form that provides sustained release of the (S)-bupropion.

### Embodiment 42

A method of enhancing the plasma levels of (R,R)-hydroxybupropion and dextromethorphan, comprising orally co-administering, at least once per day, dextromethorphan and at least about 90 mg of (S)-bupropion that is at least 95% enantiomerically pure, to a human being in need of treatment with both (R,R)-hydroxybupropion and dextromethorphan, wherein the method achieves a Cₘₐₓ of (R,R)-hydroxybupropion that is at least about 700 ng/mL in the human being, wherein the method is effective in increasing the Cₘₐₓ of (R,R)-hydroxybupropion at least 3-fold as compared the Cₘₐₓ of (R,R)-hydroxybupropion that results from administering the same of amount of (R)-bupropion to the human being.

### Embodiment 43

The method of embodiment 42, wherein the dextromethorphan and the (S)-bupropion are co-administered for at least 8 consecutive days.

### Embodiment 44

The method of embodiment 42, wherein the dextromethorphan and the (S)-bupropion are co-administered for at least 21 consecutive days.

### Embodiment 45

The method of embodiment 42, 43 or 44, wherein the dextromethorphan and the (S)-bupropion are co-administered in a single dosage form.

### Embodiment 46

The method of embodiment 42, 43, 44, or 45, wherein the dosage form is administered once daily for 1 to 3 consecutive days, then the dosage form is administered twice a day for at least the following 4 to 7 consecutive days, so that the dosage form is administered once daily or twice daily for a total of at least 8 consecutive days.

### Embodiment 47

The method of embodiment 42, 43, 44, 45, or 46, wherein the dosage form is administered once daily for 1 to 7 consecutive days, then the dosage form is administered twice a day for at least the following 7 consecutive days, so that the dosage form is administered once daily or twice daily for a total of at least 8 consecutive days.

### Embodiment 48

The method of embodiment 42, 43, 44, 45, 46, or 47, wherein (S)-bupropion is administered in a dosage form containing about 100 mg to about 110 mg of the (S)-bupropion.

### Embodiment 49

The method of embodiment 42, 43, 44, 45, 46, 47, or 48, wherein the Cₘₐₓ of (R,R)-hydroxybupropion in the human being is at least about 900 ng/mL.

### Embodiment 50

The method of embodiment 42, 43, 44, 45, 46, 47, 48, or 49, wherein the method achieves an AUC₀₋₁₂of (R,R)-hydroxybupropion in the human being that is at least about 10,000 ng·hr/mL.

### Embodiment 51

The method of embodiment 42, 43, 44, 45, 46, 47, 48, 49, or 50, wherein (S)-bupropion is administered in a dosage form that provides sustained release of the (S)-bupropion.

### Embodiment 52

A method of enhancing the plasma levels of (S)-bupropion comprising orally administering, at least once per day, at least about 90 mg of (S)-bupropion that is at least 95% enantiomerically pure, to a human being in need of treatment (S)-bupropion, wherein the method achieves a Cₘᵢₙ of (S)-bupropion that is at least about 20 ng/mL, wherein the method is effective in increasing the Cₘᵢₙ of (S)-bupropion at least 3-fold as compared the Cₘᵢₙ of (R)-bupropion that results from administering a dosage form containing the same of amount of (R)-bupropion to the human being.

### Embodiment 53

The method of embodiment 52, wherein the dextromethorphan and the (S)-bupropion are co-administered for at least 8 consecutive days.

### Embodiment 54

The method of embodiment 52, wherein the dextromethorphan and the (S)-bupropion are co-administered for at least 14 consecutive days.

### Embodiment 55

The method of embodiment 52, 53, or 54, wherein the dextromethorphan and the (S)-bupropion are co-administered in a single dosage form.

### Embodiment 56

The method of embodiment 52, 53, 54, or 55, wherein the dosage form is administered once daily for 1 to 3 consecutive days, then the dosage form is administered twice a day for at least the following 4 to 7 consecutive days, so that the dosage form is administered once daily or twice daily for a total of at least 8 consecutive days.

### Embodiment 57

The method of embodiment 52, 53, 54, 55, or 56, wherein the dosage form is administered once daily for 1 to 7 consecutive days, then the dosage form is administered twice a day for at least the following 7 consecutive days, so that the dosage form is administered once daily or twice daily for a total of at least 8 consecutive days.

### Embodiment 58

The method of embodiment 52, 53, 54, 55, 56, or 57, wherein (S)-bupropion is administered in a dosage form containing about 100 mg to about 110 mg of the (S)-bupropion.

### Embodiment 59

The method of embodiment 52, 53, 54, 55, 56, 57, or 58, wherein the method achieves a Cₘₐₓ of (S)-bupropion in the human being that is at least about 110 ng/mL.

### Embodiment 60

The method of embodiment 52, 53, 54, 55, 56, 57, 58, or 59, wherein the method achieves an AUC₀₋₁₂ of (S)-bupropion in the human being is at least about 800 ng·hr/mL.

### Embodiment 61

The method of embodiment 52, 53, 54, 55, 56, 57, 58, 59, or 60, wherein (S)-bupropion is administered in a dosage form that provides sustained release of the (S)-bupropion.

### Embodiment 62

A method of enhancing the plasma levels of (R,R)-hydroxybupropion comprising orally administering, at least once per day, at least about 90 mg of (S)-bupropion that is at least 95% enantiomerically pure, to a human being in need of treatment with (R,R)-hydroxybupropion, wherein the method achieves a Cₘᵢₙ of (R,R)-hydroxybupropion that is at least about 700 ng/mL in the human being, wherein the method is effective in increasing the Cₘᵢₙ of (R,R)-hydroxybupropion at least 3-fold as compared the Cₘᵢₙ of (R,R)-hydroxybupropion that results from administering the same of amount of (R)-bupropion to the human being.

### Embodiment 63

The method of embodiment 62, wherein the dextromethorphan and the (S)-bupropion are co-administered for at least 8 consecutive days.

### Embodiment 64

The method of embodiment 62, wherein the dextromethorphan and the (S)-bupropion are co-administered for at least 21 consecutive days.

### Embodiment 65

The method of embodiment 62, 63, or 64, wherein the dextromethorphan and the (S)-bupropion are co-administered in a single dosage form.

### Embodiment 66

The method of embodiment 62, 63, 64, or 65, wherein the dosage form is administered once daily for 1 to 3 consecutive days, then the dosage form is administered twice a day for at least the following 4 to 7 consecutive days, so that the dosage form is administered once daily or twice daily for a total of at least 8 consecutive days.

### Embodiment 67

The method of embodiment 62, 63, 64, 65, or 66, wherein the dosage form is administered once daily for 1 to 7 consecutive days, then the dosage form is administered twice a day for at least the following 7 consecutive days, so that the dosage form is administered once daily or twice daily for a total of at least 8 consecutive days.

### Embodiment 68

The method of 62, 63, 64, 65, 66, or 67, wherein (S)-bupropion is administered in a dosage form containing about 100 mg to about 110 mg of the (S)-bupropion.

### Embodiment 69

The method of embodiment 62, 63, 64, 65, 66, 66, 67, or 68, wherein the method achieves a Cₘₐₓ of (R,R)-hydroxybupropion in the human being that is at least about 900 ng/m L.

### Embodiment 70

The method of embodiment 62, 63, 64, 65, 66, 67, 68, or 69, wherein the method achieves an AUC₀₋₁₂ of (R,R)-hydroxybupropion in the human being that is at least about 10,000 ng·hr/mL.

### Embodiment 71

The method of embodiment 62, 63, 64, 65, 66, 67, 68, 69, or 70, wherein (S)-bupropion is administered in a dosage form that provides sustained release of the (S)-bupropion.

### Embodiment 72

A method of treating a central nervous system (CNS) disorder in a human being comprising administering: a dosage form comprising a therapeutically effective amount of at least about 95% enantiomerically pure (S)-bupropion, and dextromethorphan, to the human being to treat the CNS disorder.

### Embodiment 73

The method of embodiment 72, wherein the CNS disorder comprises depression.

### Embodiment 74

The method of embodiment 72, wherein the CNS disorder comprises treatment-resistant depression.

### Embodiment 75

The method of embodiment 72, wherein the CNS disorder comprises an addictive disorder.

### Embodiment 76

The method of embodiment 72, wherein the CNS disorder comprises a nicotine addiction.

### Embodiment 77

The method of embodiment 72, wherein the CNS disorder comprises an alcohol addiction.

### Embodiment 78

The method of embodiment 72, wherein the CNS disorder comprises

Alzheimer's disease.

### Embodiment 79

The method of embodiment 72, 73, 74, 75, 76, 77, or 78, wherein the dosage form is in a form of tablet, capsule, or syrup.

### Embodiment 80

The method of embodiment 72, 73, 74, 75, 76, 77, 78, or 79, wherein the dosage form is orally administered to the human being.

### Embodiment 81

The method of embodiment 72, 73, 74, 75, 76, 77, 78, 79, or 80, wherein the dosage form is orally administered to the human being daily.

### Embodiment 82

The method of embodiment 72, 73, 74, 75, 76, 77, 78, 79, 80, or 81, wherein the dosage form is orally administered to the human being once daily.

### Embodiment 83

The method of embodiment 72, 73, 74, 75, 76, 77, 78, 79, 80, 81, or 82, wherein the dosage form is orally administered to the human being twice daily.

### Embodiment 84

The method of embodiment 72, 73, 74, 75, 76, 77, 78, 79, 80, 81, 82, or 83, wherein the dosage form is orally administered to the human being under fasting conditions.

### Embodiment 85

The method of embodiment 72, 73, 74, 75, 76, 77, 78, 79, 80, 81, 82, 83, or 84, wherein the dosage form is orally administered to the human being daily for at least 8 consecutive days.

### Embodiment 86

The method of embodiment 72, 73, 74, 75, 76, 77, 78, 79, 80, 81, 82, 83, 84, or 85, wherein the dosage form contains about 100 mg to about 200 mg of the (S)-bupropion.

### Embodiment 87

The method of embodiment 72, 73, 74, 75, 76, 77, 78, 79, 80, 81, 82, 83, 84, 85, or 86, wherein the dosage form contains about 104 mg to about 106 mg of the (S)-bupropion.

### Embodiment 88

The method of embodiment 72, 73, 74, 75, 76, 77, 78, 79, 80, 81, 82, 83, 84, 85, 86, or 87, wherein the dosage form contains about 148 mg to about 152 mg of the (S)-bupropion.

### Embodiment 89

The method of embodiment 72, 73, 74, 75, 76, 77, 78, 79, 80, 81, 82, 83, 84, 85, 86, 87, or 88, wherein the both the (S)-bupropion and the dextromethorphan are in the dosage form.

### Embodiment 90

The method of embodiment 72, 73, 74, 75, 76, 77, 78, 79, 80, 81, 82, 83, 84, 85, 86, 87, 88, or 89, wherein the dosage form contains about 10 mg to about 50 mg of the dextromethorphan.

### Embodiment 91

The method of embodiment 72, 73, 74, 75, 76, 77, 78, 79, 80, 81, 82, 83, 84, 85, 86, 87, 88, 89, or 90, wherein the dosage form contains about 44 mg to about 46 mg of the dextromethorphan.

### Embodiment 92

The method of embodiment 72, 73, 74, 75, 76, 77, 78, 79, 80, 81, 82, 83, 84, 85, 86, 87, 88, 89, 90, or 91, wherein the dosage form is well tolerated.

### Embodiment 93

A method of achieving an increased plasma level of (S)-bupropion while enhancing dextromethorphan plasma levels, comprising administering a dosage form comprising a therapeutically effective amount of at least about 95% enantiomerically pure (S)-bupropion, and dextromethorphan, to a human being in need of treatment with bupropion.

### Embodiment 94

The method of embodiment 93, wherein the method is effective in achieving an increased Cₘₐₓ of (S)-bupropion as compared to administering the same amount racemic bupropion.

### Embodiment 95

The method of embodiment 93 or 94, wherein the method is effective in achieving a Cₘₐₓ of (S)-bupropion that is at least 3 times as high as the Cₘₐₓ of (R)-bupropion that results from administering a dosage form containing the same amount of (R)-bupropion to the human being.

### Embodiment 96

The method of embodiment 93, 94, or 95, wherein the method is effective in achieving an increased AUC₀₋₁₂ of (S)-bupropion as compared to administering the same amount racemic bupropion.

### Embodiment 97

The method of embodiment 96, wherein the method is effective in achieving an AUC₀₋₁₂ of (S)-bupropion that is at least 3 times as high as the AUC₀₋₁₂ of (R)-bupropion that results from administering a dosage form containing the same amount of (R)-bupropion to the human being.

### Embodiment 98

The method of embodiment 95, 96, or 97, wherein the dosage form is administered at least once a day for at least 8 consecutive days.

### Embodiment 99

The method of embodiment 95, 96, 97, or 98, wherein the dosage form is administered once daily for 1 to 3 consecutive days, then the dosage form is administered twice a day for at least the following 4 to 7 consecutive days, so that the dosage form is administered once daily or twice daily for a total of at least 8 consecutive days.

### Embodiment 100

The dosage form of embodiment 95, 96, 97, 98, or 99, wherein the dosage form is well tolerated.

### Embodiment 101

A dosage form comprising at least about 95% enantiomerically pure (R)-bupropion and dextromethorphan, wherein orally administering the dosage form to a human being provides an increased enhancement to a plasma level of dextromethorphan in the human being as compared to orally administering a reference dosage form containing the same amount of (S)-bupropion and the same amount of dextromethorphan.

### Embodiment 102

The dosage form of embodiment 101, wherein the dosage form contains about 100 mg to about 200 mg of (R)-bupropion.

### Embodiment 103

The dosage form of embodiment 102, wherein the dosage form contains about 104 mg to about 106 mg of (R)-bupropion.

### Embodiment 104

The dosage form of embodiment 102, wherein the dosage form contains about 148 mg to about 152 mg of (R)-bupropion.

### Embodiment 105

The dosage form of embodiment 101, 102, 103, or 104, wherein the dosage form contains about 10 mg to about 50 mg of dextromethorphan.

### Embodiment 106

The dosage form of embodiment 105, wherein the dosage form contains about 44 mg to about 46 mg of dextromethorphan.

### Embodiment 107

The dosage form of embodiment 101, wherein the dosage form contains about 100 mg to about 110 mg of (R)-bupropion and about 40 mg to about 50 mg of dextromethorphan.

### Embodiment 108

The dosage form of embodiment 101, 102, 103, 104, 105, 106, or 107, wherein the dosage form is orally administered to the human being daily.

### Embodiment 109

The dosage form of embodiment 101, 102, 103, 104, 105, 106, 107, or 108, wherein the dosage form is orally administered to the human being once daily.

### Embodiment 110

The dosage form of embodiment 101, 102, 103, 104, 105, 106, 107, or 108, wherein the dosage form is orally administered to the human being twice daily.

### Embodiment 111

The dosage form of embodiment 109 or 110, wherein the dosage form is orally administered to the human being daily for at least 8 consecutive days.

### Embodiment 112

The dosage form of embodiment 101, 102, 103, 104, 105, 106, 107, 108, 109, 110, or 111, wherein the dosage form is orally administered to the human being under fasting conditions.

### Embodiment 113

The dosage form of embodiment 111, wherein the dosage form achieves a Cₘₐₓ of dextromethorphan of at least about 80 ng/mL in the human being on day 8 of oral administration of the dosage form daily for 8 consecutive days.

### Embodiment 114

The dosage form of embodiment 113, wherein the dosage form is in a form of syrup, tablet, capsule, spray, or lozenge.

### Embodiment 115

The dosage form of embodiment 113 or 114, wherein the dosage form is used for treating a neuropsychiatric disorder in the human being in need thereof.

### Embodiment 116

The dosage form of embodiment 101, 102, 103, 104, 105, 106, 107, 108, 109, 110, 111, 112, 113, 114, or 115, wherein the dosage form is used for treating cold or cough in the human being in need thereof.

### Embodiment 117

The dosage form of embodiment 101, 102, 103, 104, 105, 106, 107, 108, 109, 110, 111, 112, 113, 114, or 115, wherein the dosage form is used for relieving pain in the human being in need thereof.

### Embodiment 118

The dosage form of embodiment 101, 102, 103, 104, 105, 106, 107, 108, 109, 110, 111, 112, 113, 114, or 115, wherein the dosage form is used for treatment of addiction in a human being in need thereof.

### Embodiment 119

The dosage form of embodiment 101, 102, 103, 104, 105, 106, 107, 108, 109, 110, 111, 112, 113, 114, 115, 116, 117, or 118, wherein the dosage form is well tolerated when administered to the human being.

### Embodiment 120

A method of increasing enhancement of dextromethorphan plasma level in a human being, comprising administrating a dosage form comprising at least about 95% enantiomerically pure (R)-bupropion and dextromethorphan to the human being, wherein the dosage form provides an increased enhancement to a plasma level of dextromethorphan in the human being as compared to a reference oral dosage form containing the same amount of (S)-bupropion and the same amount of dextromethorphan.

### Embodiment 121

The method of embodiment 120, wherein the dosage form contains about 100 mg to about 200 mg of (R)-bupropion.

### Embodiment 122

The method of embodiment 120, wherein the dosage form contains about 100 mg to about 110 mg of (R)-bupropion.

### Embodiment 123

The method of embodiment 120, wherein the dosage form contains about 148 mg to about 152 mg of (R)-bupropion.

### Embodiment 124

The method of embodiment 120, 121, 122, or 123, wherein the dosage form contains about 10 mg to about 50 mg of dextromethorphan.

### Embodiment 125

The method of embodiment 124, wherein the dosage form contains about 40 mg to about 50 mg of dextromethorphan.

### Embodiment 126

The method of embodiment 125, wherein the dosage form contains about 100 mg to about 110 mg of (R)-bupropion and about 40 mg to about 50 mg of dextromethorphan.

### Embodiment 127

The method of embodiment 120, 121, 122, 123, 124, 125, or 126, wherein the dosage form is orally administered to the human being under fasting conditions.

### Embodiment 128

The method of embodiment 120, 121, 122, 123, 124, 125, 126, or 127, wherein the dosage form is well tolerated.

### Embodiment 129

A method of treating a central nervous system (CNS) disorder in a human being comprising administering a dosage form comprising a therapeutically effective amount of at least about 95% enantiomerically pure (S)-bupropion to the human being to treat the CNS disorder, wherein the human being does not receive dextromethorphan.

### Embodiment 130

The method of embodiment 129, wherein the CNS disorder comprises depression.

### Embodiment 131

The method of embodiment 129 or 130, wherein the CNS disorder comprises treatment resistant depression.

### Embodiment 132

The method of embodiment 129, 130, or 131 wherein the CNS disorder is an addictive disorder.

### Embodiment 133

The method of embodiment 132, wherein the CNS disorder is nicotine addiction.

### Embodiment 134

The method of embodiment 132, wherein the CNS disorder is alcohol addiction.

### Embodiment 135

The method of embodiment 132, wherein the CNS disorder is Alzheimer's disease.

### Embodiment 136

The method of embodiment 129, 130, 131, 132, 133, 134, or 135, wherein the dosage form contains no active pharmaceutical agent other than (S)-bupropion.

### Embodiment 137

The method of embodiment 129, 130, 131, 132, 133, 134, or 135, wherein the dosage form contains less than 0.1% of any active pharmaceutical agent other than (S)-bupropion.

### Embodiment 138

The method of embodiment 129, 130, 131, 132, 133, 134, 135, 136, or 137, wherein the dosage form is in a form of a tablet, capsule, or syrup.

### Embodiment 139

The method of embodiment 129, 130, 131, 132, 133, 134, 135, 136, 137, or 138, wherein the dosage form is orally administered to the human being daily.

### Embodiment 140

The method of embodiment 139, wherein the dosage form is orally administered to the human being once daily.

### Embodiment 141

The method of embodiment 139, wherein the dosage form is orally administered to the human being twice daily.

### Embodiment 142

The method of embodiment 129, 130, 131, 132, 133, 134, 135, 136, 137, 138, 139, 140, or 141, wherein the dosage form is orally administered to the human being under fasting conditions.

### Embodiment 143

The method of embodiment 129, 130, 131, 132, 133, 134, 135, 136, 137, 138, 139, 140, 141, or 142 wherein the dosage form is orally administered to the human being daily for at least 8 consecutive days.

### Embodiment 144

The method of embodiment 129, 130, 131, 132, 133, 134, 135, 136, 137, 138, 139, 140, 141, 142, or 143, wherein the dosage form contains about 100 mg to about 200 mg of (S)-bupropion.

### Embodiment 145

The method of embodiment 144, wherein the dosage form contains about 104 mg to about 106 mg of (S)-bupropion.

### Embodiment 146

The method of embodiment 144, wherein the dosage form contains about 148 mg to about 152 mg of (S)-bupropion.

### Embodiment 147

A method of enhancing the plasma level of (S)-bupropion, comprising administering comprising administering a dosage comprising a therapeutically effective amount of at least about 95% enantiomerically pure (S)-bupropion, wherein the dosage form is free of dextromethorphan, to a human being in need of treatment with bupropion.

### Embodiment 148

The method of embodiment 147, wherein the method is effective in increasing the Cₘₐₓ of (S)-bupropion.

### Embodiment 149

The method of embodiment 147 or 148, wherein the method is effective in increasing the Cₘₐₓ of (S)-bupropion at least 3-fold as compared the Cₘₐₓ of (R)-bupropion that results from administering a dosage form containing the same of amount of (R)-bupropion to the human being.

### Embodiment 150

The method of embodiment 147, 148, or 149, wherein the method is effective in enhancing the AUC₀₋₁₂ of (S)-bupropion.

### Embodiment 151

The method of embodiment 147, 148, 149, or 150, wherein the method is effective in increasing the AUC₀₋₁₂ of (S)-bupropion at least 3-fold as compared the AUC₀₋₁₂ of (R)-bupropion that results from administering a dosage form containing the same of amount of (R)-bupropion to the human being.

### Embodiment 152

The method of embodiment 147, 148, 149, 150, or 151, wherein the dosage form is administered at least once a day for at least 8 consecutive days.

### Embodiment 153

The method of embodiment 152, wherein the oral dosage form is administered once daily for 1 to 3 consecutive days, then the dosage form is administered twice a day for at least the following 4 to 7 consecutive days, so that the dosage form is administered once daily or twice daily for a total of at least 8 consecutive days.

### Embodiment 154

The dosage form of embodiment 147, 148, 149, 150, 151, 152, or 153, wherein the dosage form is well tolerated.

### Embodiment 155

A method of enhancing the plasma levels of (S)-bupropion, comprising orally administering an oral dosage form containing at least about 90 mg of (S)-bupropion that is at least 95% enantiomerically pure, to a human being in need of treatment with (S)-bupropion and dextromethorphan, wherein the method achieves a Cₘₐₓ of (S)-bupropion that is at least about 90 ng/mL in the human being, wherein the method is effective in increasing the Cₘₐₓ of (S)-bupropion at least 3-fold as compared the Cₘₐₓ of (R)-bupropion that results from administering a dosage form containing the same of amount of (R)-bupropion to the human being, wherein the human being does not receive dextromethorphan.

### Embodiment 156

The method of embodiment 155, wherein the oral dosage form is administered for at least 8 consecutive days.

### Embodiment 157

The method of embodiment 156, wherein the oral dosage form is administered once daily for 1 to 3 consecutive days, then the dosage form is administered twice a day for at least the following 4 to 7 consecutive days, so that the dosage form is administered once daily or twice daily for a total of at least 8 consecutive days.

### Embodiment 158

The method of embodiment 155, 156, or 157, wherein the dosage form contains about 100 mg to about 110 mg of (S)-bupropion.

### Embodiment 159

The method of embodiment 155, 156, 157, or 158, wherein the Cₘₐₓ of (S)-bupropion in the human being is at least about 110 ng/mL.

### Embodiment 160

A method of enhancing the plasma levels of (R,R)-hydroxybupropion, comprising orally administering an oral dosage form containing at least about 90 mg of (S)-bupropion that is at least 95% enantiomerically pure, to a human being in need of treatment with (R,R)-hydroxybupropion and dextromethorphan, wherein the method achieves a Cₘₐₓ of (R,R)-hydroxybupropion that is at least about 90 ng/mL, wherein the method is effective in increasing the Cₘₐₓ of (R,R)-hydroxybupropion at least 3-fold as compared the Cₘₐₓ of (R,R)-hydroxybupropion that results from administering a dosage form containing the same of amount of (R)-bupropion to the human being, wherein the human being does not receive dextromethorphan.

### Embodiment 161

The method of embodiment 160, wherein the oral dosage form is administered for at least 8 consecutive days.

### Embodiment 162

The method of embodiment 161, wherein the oral dosage form is administered once daily for 1 to 3 consecutive days, then the dosage form is administered twice a day for at least the following 4 to 7 consecutive days, so that the dosage form is administered once daily or twice daily for a total of at least 8 consecutive days.

### Embodiment 163

The method of embodiment 160, 161, or 162, wherein the dosage form contains about 100 mg to about 110 mg of (S)-bupropion.

### Embodiment 164

An oral dosage form comprising (R)-bupropion in an enantiomeric excess of at least 95%, and dextromethorphan, wherein the dosage form provides an increased enhancement to a plasma level of dextromethorphan in a human being as compared to a reference oral dosage form containing the same amount of (S)-bupropion and the same amount of dextromethorphan.

### Embodiment 165

An oral dosage form comprising (R)-bupropion and dextromethorphan, wherein the dosage form can enhance plasma levels of dextromethorphan in a human being on day 1 or day 8 in a much greater extent than that of a reference oral dosage form when orally administered to the human being daily for at least 8 consecutive days, wherein the reference oral dosage form contains the same amount of bupropion as a racemic-bupropion and the same amount of dextromethorphan.

### Embodiment 166

An oral dosage form comprising (R)-bupropion and dextromethorphan, wherein the dosage form increases a mean Cₘₐₓ of dextromethorphan in a human being on day 1 by at least 150% when orally administered to the human being daily for at least 8 consecutive days, wherein the reference oral dosage form contains the same amount of bupropion as a (S)-bupropion and the same amount of dextromethorphan.

### Embodiment 167

An oral dosage form comprising (R)-bupropion and dextromethorphan, wherein the dosage form increases a mean Cₘₐₓ of dextromethorphan in a human being on day 8 by at least 25% when orally administered to the human being daily for at least 8 consecutive days, wherein the reference oral dosage form contains the same amount of bupropion as a (S)-bupropion and the same amount of dextromethorphan.

### Embodiment 168

An oral dosage form comprising (R)-bupropion and dextromethorphan, wherein the dosage form increases a mean Cₘₐₓ of dextromethorphan in a human being on day 8 by at least 20-fold as compared to that of a reference dosage form on day 1, when orally administered to the human being daily for at least 8 consecutive days, wherein the reference oral dosage form contains the same amount of bupropion as a racemic-bupropion and the same amount of dextromethorphan.

### Embodiment 169

An oral dosage form comprising (R)-bupropion and dextromethorphan, wherein the dosage form increases a mean Cₘₐₓ of dextromethorphan in a human being on day 1 by at least 60% when orally administered to the human being daily for at least 8 consecutive days, wherein the reference oral dosage form contains the same amount of bupropion as a racemic-bupropion and the same amount of dextromethorphan.

### Embodiment 170

An oral dosage form comprising (R)-bupropion and dextromethorphan, wherein the dosage form increases a mean Cₘₐₓ of dextromethorphan in a human on day 8 being by at least 20% when orally administered to the human being daily for at least 8 consecutive days, wherein the reference oral dosage form contains the same amount of bupropion as a racemic-bupropion and the same amount of dextromethorphan.

### Embodiment 171

A method of enhancing the plasma levels of dextromethorphan on day 1 or day 8, comprising orally administering a dosage form containing (R)-bupropion and dextromethorphan, in a much greater extent than orally administering a reference dosage form, when orally administered to a human being daily for at least 8 consecutive days, wherein the reference dosage form contains the same amount of bupropion as a (S)-bupropion and the same amount of dextromethorphan.

### Embodiment 172

The dosage form or method of embodiment 164, 165, 166, 167, 168, 169, 170, or 171, wherein the (R)-bupropion in the dosage form is at least 95% enantiomerically pure.

### Embodiment 173

The dosage form or method of embodiment 164, 165, 166, 167, 168, 169, 170, or 171, wherein the (S)-bupropion in the reference dosage form is at least 95% enantiomerically pure.

### Embodiment 174

The dosage form or method of embodiment 164, 165, 166, 167, 168, 169, 170, or 171, wherein the dosage form contains about 100 mg to about 150 mg of (R)-bupropion.

### Embodiment 175

The dosage form or method of embodiment 174, wherein the dosage form contains about 100 mg to about 110 mg of (R)-bupropion.

### Embodiment 176

The dosage form or method of embodiment 164, 165, 166, 167, 168, 169, 170, or 171, wherein the dosage form contains about 10 mg to about 50 mg of dextromethorphan.

### Embodiment 177

The dosage form or method of embodiment 176, wherein the dosage form contains about 40 mg to about 50 mg of dextromethorphan.

### Embodiment 178

The dosage form or method of embodiment 164, 165, 166, 167, 168, 169, 170, 171, 172, 173, 174, 175, 176, or 177, wherein the dosage form is orally administered once daily.

### Embodiment 179

The dosage form or method of embodiment 164, 165, 166, 167, 168, 169, 170, 171, 172, 173, 174, 175, 176, or 177, wherein the dosage form is orally administered twice a day.

### Embodiment 180

The dosage form or method of embodiment 178 or 179, wherein the dosage form is orally administered for at least 8 consecutive days.

### Embodiment 181

The dosage form or method of embodiment 180, wherein the dosage form contains about 100 mg to about 110 mg of (R)-bupropion and about 40 mg to about 50 mg of dextromethorphan.

### Embodiment 182

The dosage form or method of embodiment 180 or 181, wherein the oral dosage form achieves a mean Cₘₐₓ of dextromethorphan of at least about 80 ng/mL in the human being on day 8 of oral administration of the dosage form daily for 8 consecutive days.

### Embodiment 183

The dosage form or method of embodiment 180, 181, or 182, wherein the dosage form is in a form of syrup, tablet, capsule, spray, or lozenge.

### Embodiment 184

A method of treating a neuropsychiatric disorder, comprising administering a dosage form any preceding embodiment to a human being in need thereof.

### Embodiment 185

A method of treating cold or cough, comprising administering a dosage form of any preceding embodiment to a human being in need thereof.

### Embodiment 186

A method of relieving pain, comprising administering a dosage form of any preceding embodiment to a human being in need thereof.

### Embodiment 187

A method for treatment of addiction, comprising administering a dosage form of any preceding embodiment to a human being in need thereof.

### Embodiment 188

A method of treating a human being comprising orally administering a dosage form containing (S)-bupropion that is at least 95% enantiomerically pure, one or two times per day, to a human being having a condition that is treatable with (S)-bupropion, wherein the amount of (S)-bupropion administered is selected to be about 20% to about 70% of the amount of racemic bupropion that would be administered to treat the same human being for the same condition.

### Embodiment 189

A method of providing therapeutically effective plasma levels of (R,R)-hydroxybupropion comprising orally administering, one or two times per day, (S)-bupropion that is at least 95% enantiomerically pure, to a human being in need of treatment with (R,R)-hydroxybupropion, wherein (R,R)-hydroxybupropion is at least 97% of the total of amount of (R,R)-hydroxybupropion and (S,S)-hydroxybupropion present in the plasma of the human being, and wherein the method achieves a Cₘₐₓ of (R,R)-hydroxybupropion that is at least about 500 ng/mL in the human being.

### Embodiment 190

A method of treating a human being comprising orally administering a dosage form containing (S)-bupropion that is at least 95% enantiomerically pure to a human being in need of treatment with (S)-bupropion, wherein the (S)-bupropion is the sole active agent used to treat the human being.

### Embodiment 191

A method of treating a human being comprising orally administering a dosage form containing about 50 mg to about 100 mg of (S)-bupropion that is at least 95% enantiomerically pure, one or two times per day, to a human being in need of treatment with (S)-bupropion.

### Embodiment 192

A method of treating a human being comprising orally administering a dosage form containing (S)-bupropion that is at least 95% enantiomerically pure, one or two times per day, to a human being in need of treatment with (S)-bupropion, wherein the method achieves a Cₘₐₓ of (S)-bupropion that is at least about 60 ng/mL.

### Embodiment 193

A method of treating a human being comprising orally administering a dosage form containing (S)-bupropion that is at least 95% enantiomerically pure, one or two times per day, to a human being in need of treatment with (S)-bupropion, wherein (R,R)-hydroxybupropion is at least 97% of the total of amount of (R,R)-hydroxybupropion and (S,S)-hydroxybupropion present in the plasma of the human being.

### Embodiment 194

A method of providing therapeutically effective plasma levels of (R,R)-hydroxybupropion comprising orally administering, one or two times per day, (S)-bupropion that is at least 95% enantiomerically pure, to a human being in need of treatment with (R,R)-hydroxybupropion, wherein (R,R)-hydroxybupropion is at least 97% of the total of amount of (R,R)-hydroxybupropion and (S,S)-hydroxybupropion present in the plasma of the human being.

### Embodiment 195

A method of providing therapeutically effective plasma levels of (R,R)-hydroxybupropion comprising orally administering, one or two times per day, (S)-bupropion that is at least 95% enantiomerically pure, to a human being in need of treatment with (R,R)-hydroxybupropion, wherein the method achieves a Cₘₐₓ of (R,R)-hydroxybupropion that is at least about 500 ng/mL in the human being.

### Embodiment 196

The method of any preceding embodiment, such as embodiments 184, 185, 186, 187, 188, 189, 190, 191, 192, 193, 194, or 195, wherein dextromethorphan is not administered to the human being.

### Embodiment 197

A method of treating a human being comprising orally administering a dosage form containing (S)-bupropion that is at least 95% enantiomerically pure, one or two times per day, to a human being in need of treatment with (S)-bupropion, wherein the method achieves a Cₘₐₓ of (S)-bupropion that is at least about 70 ng/mL.

### Embodiment 198

A method of treating a human being comprising orally administering a dosage form containing (S)-bupropion that is at least 95% enantiomerically pure, one or two times per day, to a human being in need of treatment with (S)-bupropion, wherein the method achieves an AU₀₋₁₂ of (S)-bupropion that is at least about 600 ng·h/mL.

### Embodiment 199

A method of treating a human being comprising orally administering a dosage form containing (S)-bupropion that is at least 95% enantiomerically pure, one or two times per day, to a human being in need of treatment with (S)-bupropion, wherein the method achieves a Cₘₐₓ of (R,R)-hydroxybupropion that is at least about 800 ng/mL.

### Embodiment 200

A method of treating a human being comprising orally administering a dosage form containing (S)-bupropion that is at least 95% enantiomerically pure, one or two times per day, to a human being in need of treatment with (S)-bupropion, wherein the method achieves an AUC₀₋₁₂ of (R,R)-hydroxybupropion that is at least about 8,000 ng·h/mL.

### Embodiment 201

A method of treating a human being comprising orally administering a dosage form containing (S)-bupropion that is at least 95% enantiomerically pure, one or two times per day, to a human being in need of treatment with (S)-bupropion, wherein the method achieves a Cₘₐₓ of erythrohydroxybupropion that is at least about 90 ng/mL.

### Embodiment 202

A method of treating a human being comprising orally administering a dosage form containing (S)-bupropion that is at least 95% enantiomerically pure, one or two times per day, to a human being in need of treatment with (S)-bupropion, wherein the method achieves an AUC₀₋₁₂ of erythrohydroxybupropion that is at least about 1,000 ng·h/mL.

### Embodiment 203

A method of treating a human being comprising orally administering a dosage form containing (S)-bupropion that is at least 95% enantiomerically pure, one or two times per day, to a human being in need of treatment with (S)-bupropion, wherein the method achieves a Cₘₐₓ of threohydroxybupropion that is at least about 450 ng/mL.

### Embodiment 204

A method of treating a human being comprising orally administering a dosage form containing (S)-bupropion that is at least 95% enantiomerically pure, one or two times per day, to a human being in need of treatment with (S)-bupropion, wherein the method achieves an AUC₀₋₁₂ of threohydroxybupropion that is at least about 5,000 ng·h/mL.

### Embodiment 205

A dosage form comprising (S)-bupropion which is at least 95% enantiomerically pure.

### Embodiment 206

The dosage form of embodiment 205, which contains about 100 mg to about 200 mg of (S)-bupropion.

### Embodiment 207

The dosage form of any preceding embodiment, wherein the dosage form contains less than 0.1% of any other active pharmaceutical agent.

### Embodiment 208

The dosage form of any preceding embodiment, further comprising dextromethorphan.

### Embodiment 209

The dosage form of embodiment 208, wherein the dosage form contains about 10 mg to about 50 mg of dextromethorphan.

### Embodiment 210

A method of enhancing the plasma level of (S)-bupropion or a metabolite thereof, comprising administering a dosage form of embodiment 205, 206, 207, 208, or 209 to a human being in need of treatment with bupropion or a metabolite thereof.

### Embodiment 211

The method of any preceding embodiment, wherein the method is effective in enhancing the Cₘₐₓ of (S)-bupropion.

### Embodiment 212

The method of any preceding embodiment, wherein the method is effective in increasing the Cₘₐₓ of (S)-bupropion at least 3-fold as compared the Cₘₐₓ of (R)-bupropion that results from administering a dosage form containing the same of amount of (R)-bupropion to the human being.

### Embodiment 213

The method of any preceding embodiment, wherein the method is effective in enhancing the AUC₀₋₁₂of (S)-bupropion.

### Embodiment 214

The method of any preceding embodiment, wherein the method is effective in increasing the AUC₀₋₁₂ of (S)-bupropion at least 3-fold as compared the AUC₀₋₁₂ of (R)-bupropion that results from administering a dosage form containing the same of amount of (R)-bupropion to the human being.

### Embodiment 215

The method of any preceding embodiment, wherein the method is effective in enhancing the Cₘₐₓ of (R,R)-hydroxybupropion.

### Embodiment 216

The method of any preceding embodiment, wherein the method is effective in increasing the Cₘₐₓ of (R,R)-hydroxybupropion at least 3-fold as compared the Cₘₐₓ of (R,R)-hydroxybupropion that results from administering a dosage form containing the same of amount of (R)-bupropion to the human being.

### Embodiment 217

The method of any preceding embodiment, wherein the method is effective in enhancing the AUC₀₋₁₂ of (R,R)-hydroxybupropion.

### Embodiment 218

The method of embodiment 13, wherein the method is effective in increasing the AUC₀₋₁₂of (R,R)-hydroxybupropion at least 3-fold as compared the AUC₀₋₁₂of (R,R)-hydroxybupropion that results from administering a dosage form containing the same of amount of (R)-bupropion to the human being.

### Embodiment 219

The method of any preceding embodiment, wherein the dosage form is administered at least once a day for at least 8 consecutive days.

### Embodiment 220

A method of treating a neurological condition, comprising administering a dosage form of any preceding embodiment to a human being in need thereof.

### Embodiment 221

The method of embodiment 16, wherein the neurological condition is depression.

### Embodiment 222

A method of increasing the plasma levels of dextromethorphan comprising administering a combination of (R)-bupropion and dextromethorphan to a human being in need of treatment by dextromethorphan, wherein the (R)-bupropion is at least 95% enantiomerically pure.

### Embodiment 223

The method of embodiment 18, wherein the Cₘₐₓ of dextromethorphan is increased by at least 20% as compared to administering the same amount of (S)-bupropion and the same amount of dextromethorphan.

### Embodiment 224

A method of delivering (S)-bupropion to the plasma of a human being comprising: orally administering a dosage form containing (S)-bupropion that is at least 95% enantiomerically pure to the human being.

### Embodiment 225

A method of delivering both (R)-bupropion and (S)-bupropion to the plasma of a human being comprising: orally administering a dosage form containing (S)-bupropion that is at least 95% enantiomerically pure to the human being, wherein the Cₘₐₓ of (R)-bupropion is within 20% of the Cₘₐₓ of (R)-bupropion that would result from administering the same amount of racemic bupropion to the human being.

### Embodiment 226

A method of delivering a bupropion to the plasma of a human being comprising: orally administering a dosage form containing (S)-bupropion that is at least 95% enantiomerically pure to the human being, wherein the AUC₀₋₁₂ of (R)-bupropion is within 20% of the AUC₀₋₁₂ of (R)-bupropion that would result from administering the same amount of racemic bupropion to the human being.

### Embodiment 227

A method of delivering a bupropion to the plasma of a human being comprising: orally administering a dosage form containing (S)-bupropion that is at least 95% enantiomerically pure to the human being, wherein the Cₘₐₓ of (R,R)-hydroxybupropion is within 20% of the Cₘₐₓ of (R,R)-hydroxybupropion that would result from administering the same amount of racemic bupropion to the human being.

### Embodiment 228

The method of embodiment 224, 225, 226, or 227 wherein the (S)-bupropion is administered for at least 8 consecutive days.

### Embodiment 229

The method of embodiment 228, wherein the (S)-bupropion is administered for at least 14 consecutive days.

### Embodiment 230

The method of embodiment 224, 225, 226, 227, 228, or 229, wherein the dosage form contains about 50 mg to about 150 mg of (S)-bupropion.

### Embodiment 231

The method of embodiment 224, 225, 226, 227, 228, or 229, wherein the dosage form contains about 40 mg to about 90 mg of (S)-bupropion.

### Embodiment 232

The method of any preceding embodiment, wherein administering the dosage form results in a combined Cₘₐₓ of (S)-bupropion and (R)-bupropion, on day 8, that is at least about 100 ng/mL.

### Embodiment 233

The method of any preceding embodiment, wherein administering the dosage form results in a combined AUC₀₋₁₂ of (S)-bupropion and (R)-bupropion, on day 8, that is at least about 800 ng·hr/mL.

### Embodiment 234

The method of any preceding embodiment, wherein administering the dosage form results in a combined Cₘₐₓ of (S,S)-hydroxybupropion and (R,R)-hydroxybupropion, on day 8, that is at least about 1,000 ng/mL.

### Embodiment 235

The method of any preceding embodiment, wherein administering the dosage form results in a combined AUC₀₋₁₂ of (S,S)-hydroxybupropion and (R,R)-hydroxybupropion, on day 8, that is at least about 10,000 ng·hr/mL.

### Embodiment 236

The method of any preceding embodiment, wherein administering the dosage form results in a Cₘₐₓ of erythrohydroxybupropion, on day 8, that is at least about 100 ng/m L.

### Embodiment 237

The method of any preceding embodiment, wherein administering the dosage form results in a AUC₀₋₁₂ of erythrohydroxybupropion, on day 8, that is at least about 1,500 ng·hr/mL.

### Embodiment 238

The method of any preceding embodiment, wherein administering the dosage form results in a Cₘₐₓ of threohydroxybupropion, on day 8, that is at least about 600 ng/m L.

### Embodiment 239

The method of any preceding embodiment, wherein administering the dosage form results in a combined AUC₀₋₁₂ of threohydroxybupropion, on day 8, that is at least about 5,000 ng·hr/mL.

Unless otherwise indicated, all numbers expressing quantities of ingredients, properties such as amounts, AUC values, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A method of delivering hydroxybupropion to the plasma of a human being comprising:
orally administering a dosage form containing (S)-bupropion that is at least 95% enantiomerically pure to the human being so that the human being receives a first amount of (S)-bupropion, wherein the Cₘₐₓ of hydroxybupropion is within 20% of the Cₘₐₓ of hydroxybupropion that would result from administering racemic bupropion to the human being in an amount that is twice the first amount of (S)-bupropion.

2. The method of claim 1, wherein the dosage form is administered for at least 8 consecutive days.

3. The method of claim 1, wherein the dosage form is administered for at least 14 consecutive days.

4. The method of claim 1, wherein the dosage form contains about 50 mg to about 150 mg of (S)-bupropion.

5. The method of claim 2, wherein the dosage form contains about 40 mg to about 90 mg of (S)-bupropion.

6. The method of claim 5, wherein administering the dosage form results in a combined Cₘₐₓ of (S)-bupropion and (R)-bupropion, on day 8, that is at least about 100 ng/mL.

7. The method of claim 5, wherein administering the dosage form results in a combined AUC₀₋₁₂ of (S)-bupropion and (R)-bupropion, on day 8, that is at least about 800 ng·hr/mL.

8. The method of claim 5, wherein administering the dosage form results in a combined Cₘₐₓ of (S,S)-hydroxybupropion and (R,R)-hydroxybupropion, on day 8, that is at least about 1,000 ng/m L.

9. The method of claim 5, wherein administering the dosage form results in a combined AUC₀₋₁₂ of (S,S)-hydroxybupropion and (R,R)-hydroxybupropion, on day 8, that is at least about 10,000 ng·hr/mL.

10. The method of claim 5, wherein administering the dosage form results in a combined Cₘₐₓ of erythrohydroxybupropion, on day 8, that is at least about 100 ng/mL.

11. The method of claim 5, wherein administering the dosage form results in a combined AUC₀₋₁₂ of erythrohydroxybupropion, on day 8, that is at least about 1,500 ng·hr/mL.

12. The method of claim 5, wherein administering the dosage form results in a combined Cₘₐₓ of threohydroxybupropion, on day 8, that is at least about 600 ng/mL.

13. The method of claim 5, wherein administering the dosage form results in a combined AUC₀₋₁₂ of threohydroxybupropion, on day 8, that is at least about 5,000 ng·hr/mL.

14. A method of delivering both (R)-bupropion and (S)-bupropion to the plasma of a human being comprising: orally administering a dosage form containing (S)-bupropion that is at least 95% enantiomerically pure to the human being so that the human being receives a first amount of (S)-bupropion, wherein the Cₘₐₓ of (R)-bupropion is within 20% of the Cₘₐₓ of (R)-bupropion that would result from administering racemic bupropion to the human being in an amount that is twice the first amount of (S)-bupropion.

15. The method of claim 14, wherein the dosage form is administered for at least 8 consecutive days.

16. The method of claim 14, wherein the dosage form is administered for at least 14 consecutive days.

17. The method of claim 14, wherein the dosage form contains about 50 mg to about 150 mg of (S)-bupropion.

18. The method of claim 15, wherein the dosage form contains about 40 mg to about 90 mg of (S)-bupropion.

19. The method of claim 18, wherein administering the dosage form results in a combined Cₘₐₓ of (S)-bupropion and (R)-bupropion, on day 8, that is at least about 100 ng/mL.

20. The method of claim 18, wherein administering the dosage form results in a combined AUC₀₋₁₂ of (S)-bupropion and (R)-bupropion, on day 8, that is at least about 800 ng·hr/mL.

21. The method of claim 18, wherein administering the dosage form results in a combined Cₘₐₓ of (S,S)-hydroxybupropion and (R,R)-hydroxybupropion, on day 8, that is at least about 1,000 ng/m L.

22. The method of claim 18, wherein administering the dosage form results in a combined AUC₀₋₁₂ of (S,S)-hydroxybupropion and (R,R)-hydroxybupropion, on day 8, that is at least about 10,000 ng·hr/mL.

23. The method of claim 18, wherein administering the dosage form results in a combined Cₘₐₓ of erythrohydroxybupropion, on day 8, that is at least about 100 ng/mL.

24. The method of claim 18, wherein administering the dosage form results in a combined AUC₀₋₁₂ of erythrohydroxybupropion, on day 8, that is at least about 1,500 ng·hr/mL.

25. The method of claim 18, wherein administering the dosage form results in a combined Cₘₐₓ of threohydroxybupropion, on day 8, that is at least about 600 ng/mL.

26. The method of claim 18, wherein administering the dosage form results in a combined AUC₀₋₁₂ of threohydroxybupropion, on day 8, that is at least about 5,000 ng·hr/mL.
